(19) United States
(12) Patent Application Publication
(10) Pub. No.: US 2025/0268591 A1
(43) Pub. Date: Aug. 28, 2025

(54) TISSUE ANCHORS AND DELIVERY SYSTEMS

(71) Applicant: Siesta Medical, Inc., Los Gatos, CA (US)

(72) Inventors: Erik van der Burg, Los Gatos, CA (US); Peter Martin, Mountain View, CA (US); Jasper Jackson, Newark, CA (US)

(21) Appl. No.: 19/022,262

(22) Filed: Jan. 15, 2025

Related U.S. Application Data
(60) Provisional application No. 63/550,249, filed on Feb. 6, 2024.

(51) Int. Cl. A61B 17/04; A61F 5/56

(52) U.S. Cl. CPC A61B 17/0401; A61B 2017/0409; A61B 2017/0445; A61B 2017/0464; A61F 5/566

(57) ABSTRACT

The tissue anchor assembly can include a tissue anchor comprising an elongated member comprising a first end and a second end. The elongated member can include a lumen extending from the first end to the second end. The elongated member can include an opening between the first end and the second end. The tissue anchor assembly can include a suture. In some embodiments, the suture enters the lumen at the first end of the elongated member, forms a suture loop through the opening of the elongated member, and exits the lumen at the second end of the elongated member. The tissue anchor delivery system can be provided. Methods of use can be provided.

### PRIORITY CLAIM

The present application claims priority benefit under 35 U.S.C. § 119 (e) of U.S. Provisional Patent Application No. 63/550,249 filed on Feb. 6, 2024, the disclosures of which is incorporated by reference herein in its entirety.

### BACKGROUND

### Field

In some aspects, this disclosure relates generally to tissue anchors for suspending sutures within tissue and methods for tissue suspension. In some aspects, this disclosure relates generally to delivery systems for delivering tissue anchors. In some aspects, the systems and methods are disclosed for tongue suspension using a tissue anchor and bone anchor for treating obstructive sleep apnea.

### Description of the Related Art

In many surgical procedures, there is a need to pass a suture deep into tissue. Sometimes, a surgeon needs to pass a suture deep into tissue to suspend the tissue by fixing the suture to bone. In particular, one such surgical procedure is suspension of the genioglossus muscle of the tongue for treating conditions such as obstructive sleep apnea (OSA).

Respiratory disorders during sleep are recognized as a common disorder with significant clinical consequences. During the various stages of sleep, the human body exhibits different patterns of brain and muscle activity. In particular, the REM sleep stage is associated with reduced or irregular ventilatory responses to chemical and mechanical stimuli and a significant degree of muscle inhibition. This muscle inhibition may lead to relaxation of certain muscle groups, including but not limited to muscles that maintain the patency of the upper airways, and create a risk of airway obstruction during sleep. Because muscle relaxation narrows the lumen of the airway, greater inspiratory effort may be required to overcome airway resistance. This increased inspiratory effort paradoxically increases the degree of airway resistance and obstruction through a Bernoulli effect on the flaccid pharyngeal walls during REM sleep.

Obstructive Sleep Apnea (OSA) is a sleep disorder that affects up to 2% to 4% of the population in the United States. OSA is characterized by an intermittent cessation of airflow in the presence of continued inspiratory effort. When these obstructive episodes occur, an affected person will transiently arouse, regain muscle tone and reopen the airway. Because these arousal episodes typically occur 10 to 60 times per night, sleep fragmentation occurs which produces excessive daytime sleepiness. Some patients with OSA experience over 100 transient arousal episodes per hour.

In addition to sleep disruption, OSA may also lead to cardiovascular and pulmonary disease. Apnea episodes of 60 seconds or more have been shown to decrease the partial pressure of oxygen in the lung alveoli by as much as 35 to 50 mm Hg. Some studies suggest that increased catecholamine release in the body due to the low oxygen saturation causes increases in systemic arterial blood pressure, which in turn causes left ventricular hypertrophy and eventually left heart failure. OSA is also associated with pulmonary hypertension, which can result in right heart failure.

Radiographic studies have shown that the site of obstruction in OSA is isolated generally to the supralaryngeal airway, but the particular site of obstruction varies with each person and multiple sites may be involved. A small percentage of patients with OSA have obstructions in the nasopharynx caused by deviated septums or enlarged turbinates. These obstructions may be treated with septoplasty or turbinate reduction procedures, respectively. More commonly, the oropharynx and the hypopharynx are implicated as sites of obstruction in OSA. Some studies have reported that the occlusion begins with the tongue falling back in an anterior-posterior direction (A-P) to contact with the soft palate and posterior pharyngeal wall, followed by further occlusion of the lower pharyngeal airway in the hypopharynx. This etiology is consistent with the physical findings associated with OSA, including a large base of tongue, a large soft palate, shallow palatal arch and a narrow mandibular arch. Other studies, however, have suggested that increased compliance of the lateral walls of the pharynx contributes to airway collapse. In the hypopharynx, radiographic studies have reported that hypopharyngeal collapse is frequently caused by lateral narrowing of the pharyngeal airway, rather than narrowing in the A-P direction.

### SUMMARY

There remains a need for improved methods and devices for treating various conditions, including but not limited to obstructive sleep apnea. There is also a need for improved devices and methods for delivering a suture into tissue, anchoring the suture with a tissue anchor, and anchoring the suture to bone with a bone anchor. Specifically with respect to current methods for tongue suspension, there is a need to prevent the suture from pulling out of tissue. There is also a need to reduce infection risk due to suture exposure to the oral cavity. There is also a need to improve the surgeon's range and ability to precisely locate and orient the tissue anchor and corresponding suture. Further, there is a need to improve the ability of surgeons to properly tension the implanted suture loop while simultaneously controlling the final tension of the suture loop.

In some embodiments, a tissue anchor assembly is provided. The tissue anchor assembly can include a tissue anchor comprising an elongated member. In some embodiments, the elongated member comprises a first end and a second end. In some embodiments, the elongated member comprises a lumen extending from the first end to the second end. In some embodiments, the elongated member comprises an opening between the first end and the second end. The tissue anchor assembly can include a suture. In some embodiments, the suture enters the lumen at the first end of the elongated member, forms a suture loop through the opening of the elongated member, and exits the lumen at the second end of the elongated member.

In some embodiments, the suture is a continuous suture. In some embodiments, the suture comprises a suture tail extending from the first end of the elongated member. In some embodiments, the suture tail is configured to act as the release suture to remove the tissue anchor. In some embodiments, the tissue anchor assembly can include a bone anchor. In some embodiments, the suture loop is configured to be secured to the bone anchor. In some embodiments, the bone anchor is configured to be implanted on a mandible of a patient. In some embodiments, the suture comprises a knot near the second end of the elongated member. In some embodiments, the knot is an overhand knot. In some embodiments, the suture comprises a knot near the first end of the elongated member. In some embodiments, the knot is an overhand knot. In some embodiments, the suture comprises a knot along the suture loop. In some embodiments, the knot is an overhand knot. In some embodiments, the knot is spaced from the opening of the elongated member.

In some embodiments, a tissue anchor delivery system is provided. The tissue anchor delivery system can include a tissue anchor comprising an elongated member and a suture. The tissue anchor delivery system can include a hypo tube handle. The tissue anchor delivery system can include a hypo tube coupled the hypo tube handle. In some embodiments, the hypo tube is configured to receive the elongated member. The tissue anchor delivery system can include a push rod handle. The tissue anchor delivery system can include a push rod coupled to the push rod handle. In some embodiments, the push rod is disposed at least partially within the hypo tube. In some embodiments, the push rod is configured to advance the elongated member from the hypo tube. In some embodiments, a stop disposed between the hypo tube handle and the push rod handle.

In some embodiments, the hypo tube handle comprises a suture cleat. In some embodiments, the stop is configured to be removed to allow the push rod handle to advance toward the hypo tube handle to advance the elongated member from the hypo tube.

In some embodiments, a method is provided. The method can include advancing an elongated member of a tissue anchor into tissue. In some embodiments, the elongated member is coupled to a suture. In some embodiments, the suture enters a lumen at a first end of the elongated member, forms a suture loop through an opening between the first end and a second end of the elongated member, and exits the lumen at the second end of the elongated member. The method can include securing the suture loop to a bone anchor.

In some embodiments, the method can include securing the bone anchor to a mandible of a patient. In some embodiments, a suture tail extending from the first end of the elongated member acts as a release suture to remove the tissue anchor.

In some embodiments, a tissue anchor delivery system is provided. The tissue anchor delivery system can include a tissue anchor comprising an elongated member and a suture. The tissue anchor delivery system can include a hypo tube handle. The tissue anchor delivery system can include a hypo tube coupled the hypo tube handle. In some embodiments, the hypo tube is configured to receive the elongated member. The tissue anchor delivery system can include a push rod handle. The tissue anchor delivery system can include a push rod coupled to the push rod handle. In some embodiments, the push rod is disposed at least partially within the hypo tube. In some embodiments, the push rod is configured to advance the elongated member from the hypo tube.

In some embodiments, the tissue anchor delivery system can include a nose cone comprising a ramp. In some embodiments, the nose cone and the hypo tube are coupled. In some embodiments, the hypo tube comprises an opening, wherein the elongated member is deployed through the opening. In some embodiments, the elongated member comprises a chamfer on a leading edge. In some embodiments, the suture is configured to be constrained relative to the hypo tube handle. In some embodiments, the push rod comprises a chamfer. In some embodiments, the tissue anchor delivery system can include a nose cone coupled to the hypo tube. In some embodiments, the hypo tube handle comprises a track. In some embodiments, the hypo tube handle comprises a suture trap. In some embodiments, the tissue anchor delivery system can include a suture trap. In some embodiments, the push rod handle is configured to latch with the suture trap for delivery of the tissue anchor. In some embodiments, the tissue anchor delivery system can include a suture trap. In some embodiments, the push rod handle is configured to advance to release the suture trap. In some embodiments, the tissue anchor delivery system can include a suture trap. In some embodiments, the push rod handle is configured to advance to deploy the tissue anchor from the hypo tube.

In some embodiments, a tissue anchor assembly is provided. The tissue anchor assembly can include a tissue anchor comprising an elongated member. In some embodiments, the elongated member comprises a first end and a second end. In some embodiments, the elongated member comprises a lumen extending from the first end, wherein the elongated member comprises an opening. The tissue anchor assembly can include a suture.

In some embodiments, the suture enters the lumen at the first end of the elongated member and exits the lumen at the opening. In some embodiments, the suture enters the lumen at the first end of the elongated member, forms a loop through the opening, and exits the lumen at the second end. In some embodiments, the suture comprises a knot near the first end. In some embodiments, the suture comprises a knot near the opening. In some embodiments, the suture comprises a knot near the second end. In some embodiments, the elongated member comprises a longitudinal rib. In some embodiments, the elongated member comprises an undercut. In some embodiments, the elongated member comprises a row of barbs. In some embodiments, the elongated member comprises a fin. In some embodiments, the elongated member comprises a spiral. In some embodiments, a through axis of the opening is perpendicular to the longitudinal axis of the elongated member. In some embodiments, the opening is perpendicular to the lumen.

### BRIEF DESCRIPTION

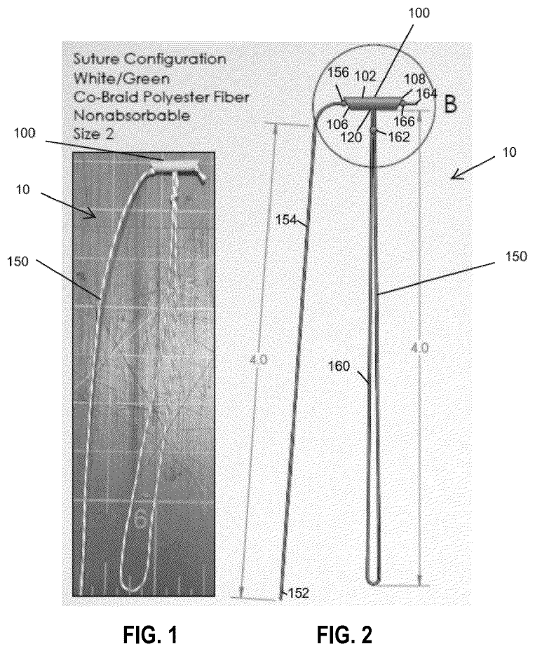

FIG. 1 illustrates an embodiment of a tissue anchor and a suture.

FIG. 2 illustrates a schematic view of the tissue anchor and the suture of FIG. 1.

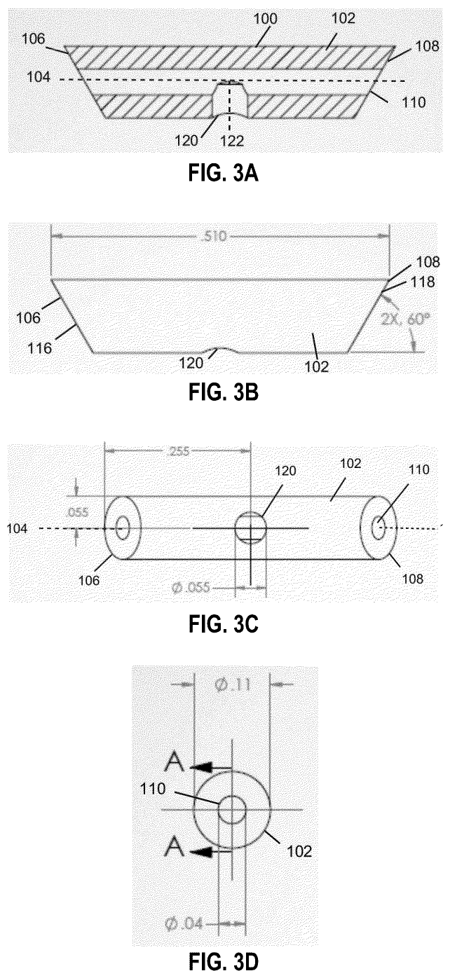

FIGS. 3A-3D illustrate views of the tissue anchor of FIG. 1.

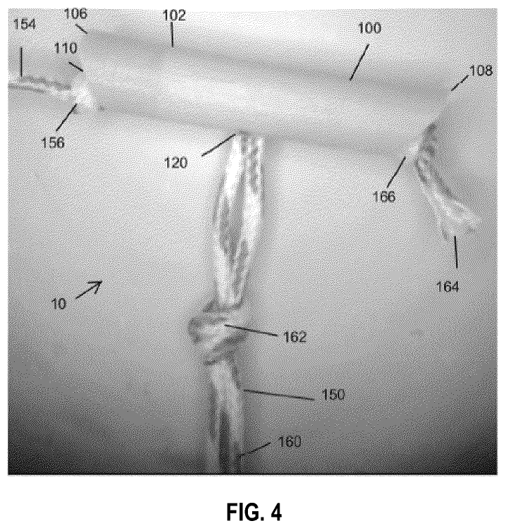

FIG. 4 illustrates a view of the tissue anchor and the suture of FIG. 1.

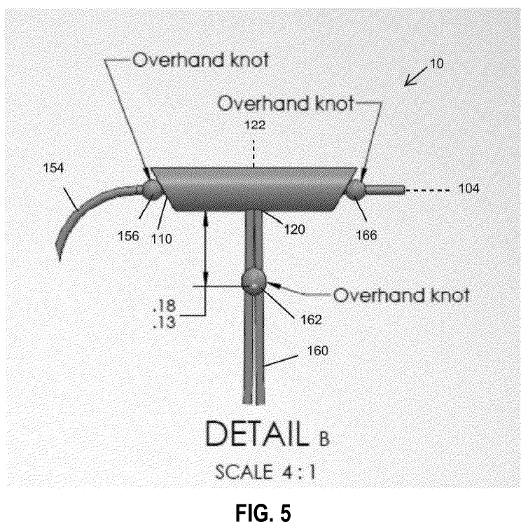

FIG. 5 illustrates a schematic view of the tissue anchor and the suture of FIG. 1.

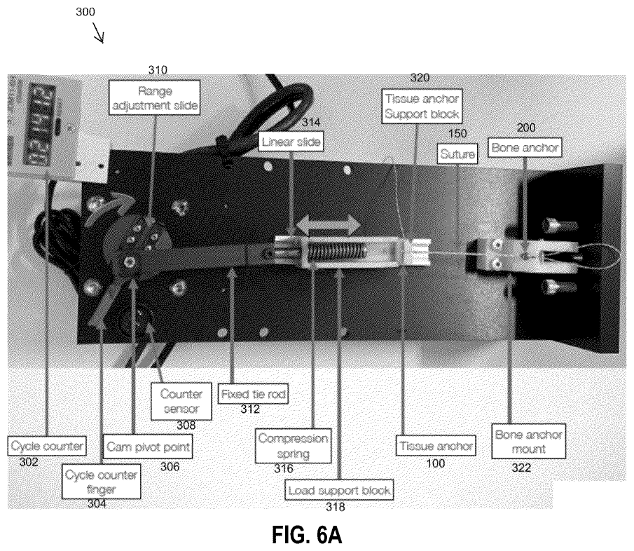

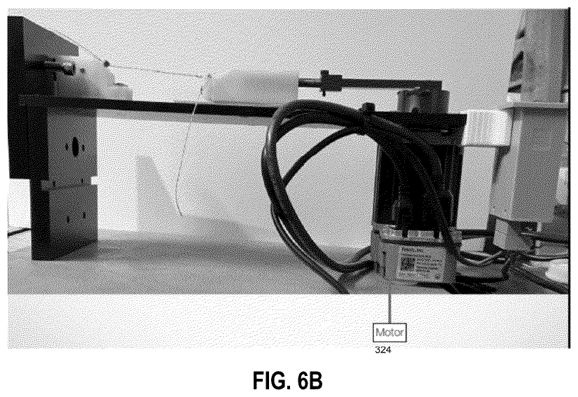

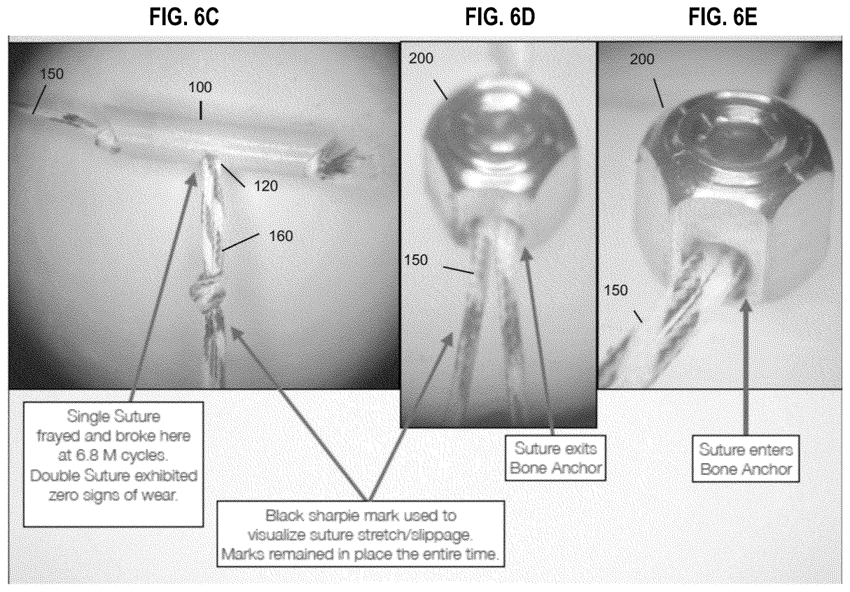

FIGS. 6A-6E are view of an embodiment of the tissue anchor and the suture of FIG. 1 and a bone anchor for fatigue testing.

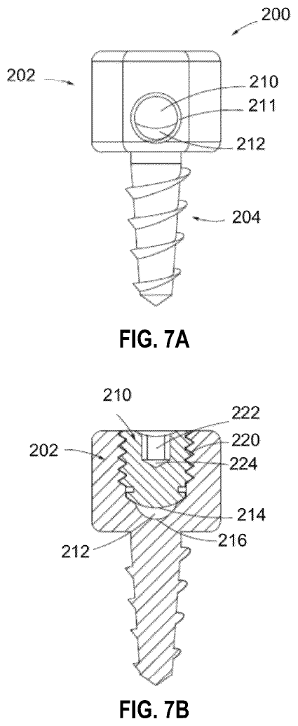

FIGS. 7A-7B are view of an embodiment of a bone anchor.

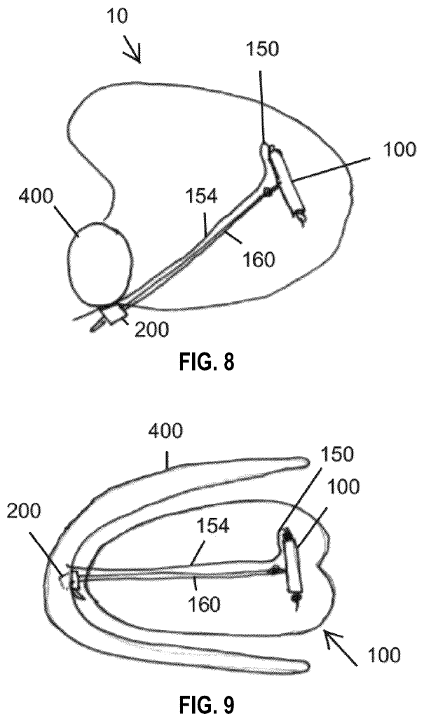

FIG. 8 illustrates an embodiment of a tissue anchor assembly in relation to a mandible.

FIG. 9 illustrates an embodiment of the tissue anchor assembly in relation to the mandible.

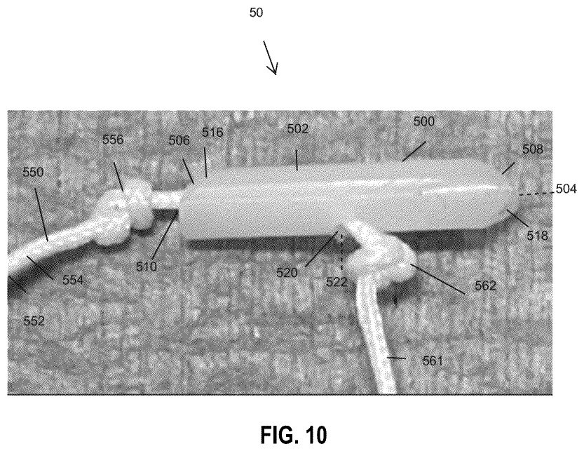

FIG. 10 illustrates a view of an embodiment of a tissue anchor and a suture.

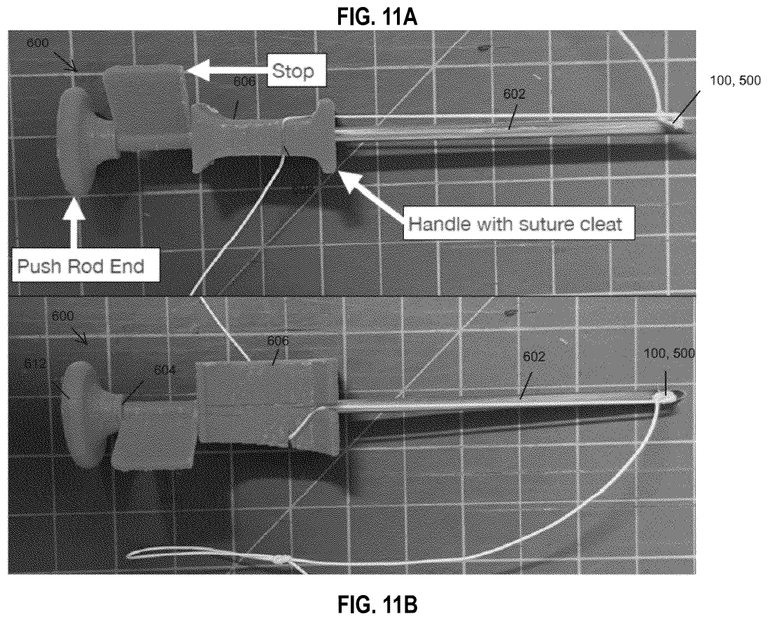

FIGS. 11A-11B illustrates an embodiment of a delivery system.

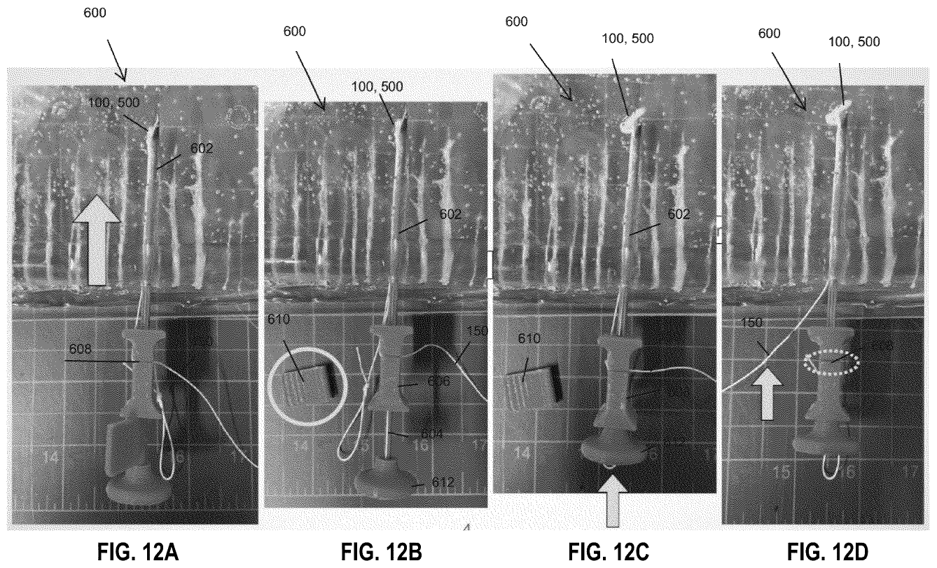

FIGS. 12A-12D illustrate a method of deployment using the delivery system of FIG. 11A.

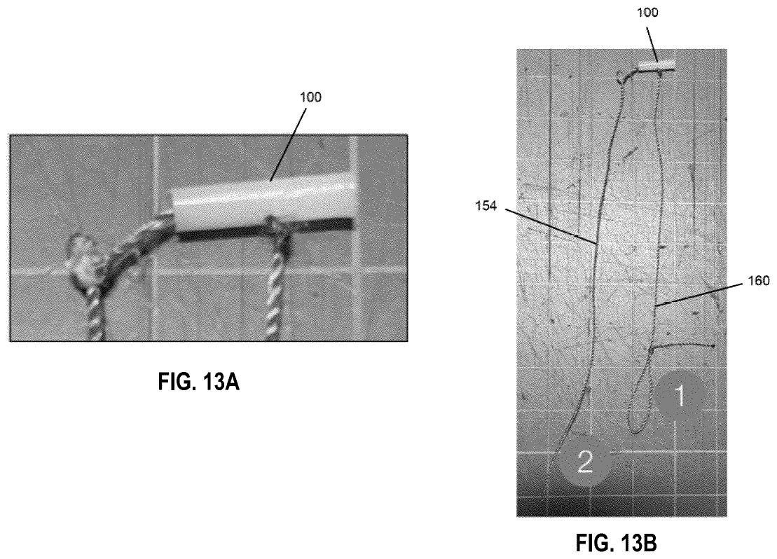

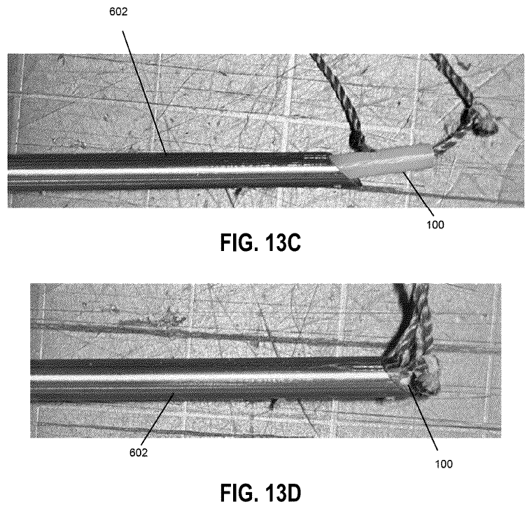

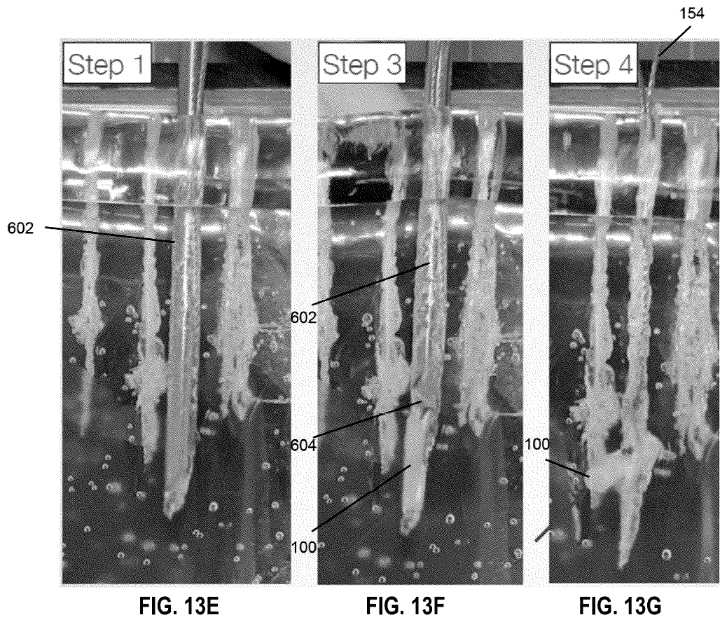

FIGS. 13A-13G illustrate a method of deployment.

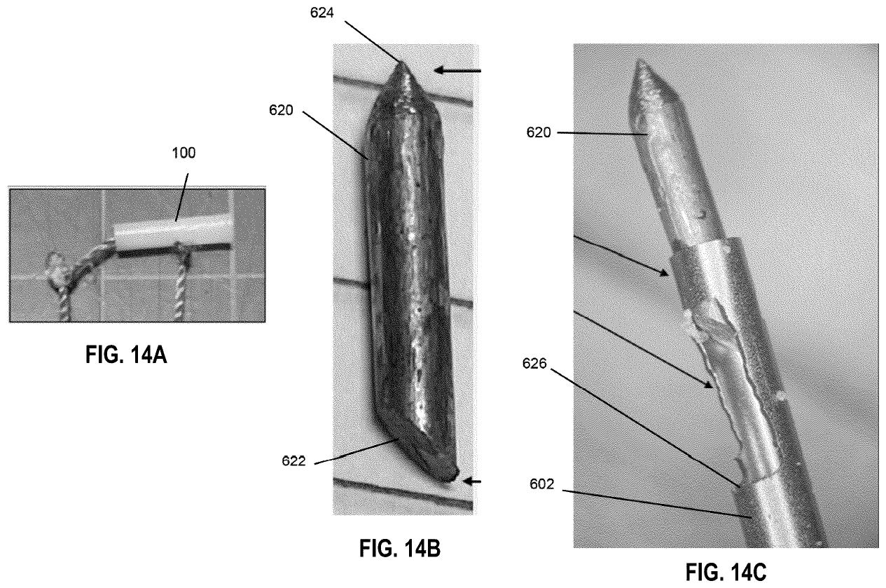

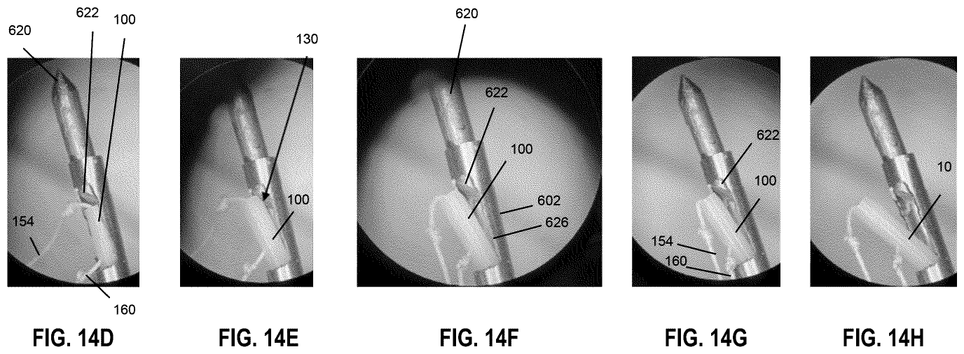

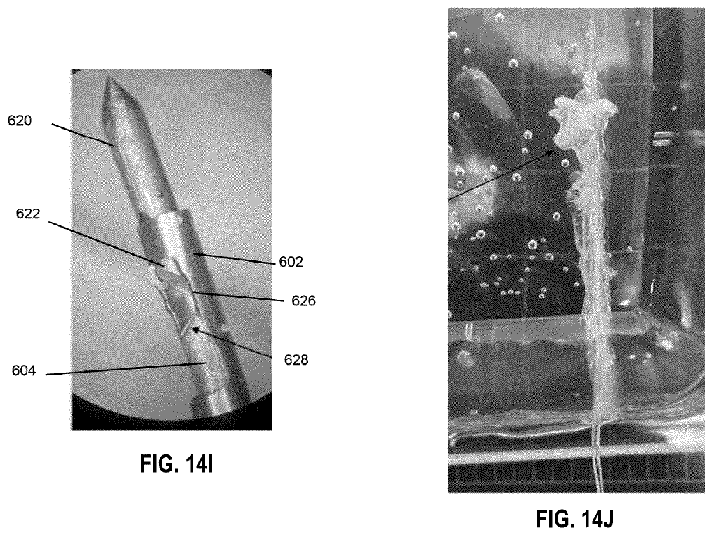

FIGS. 14A-14J illustrate features of the delivery system.

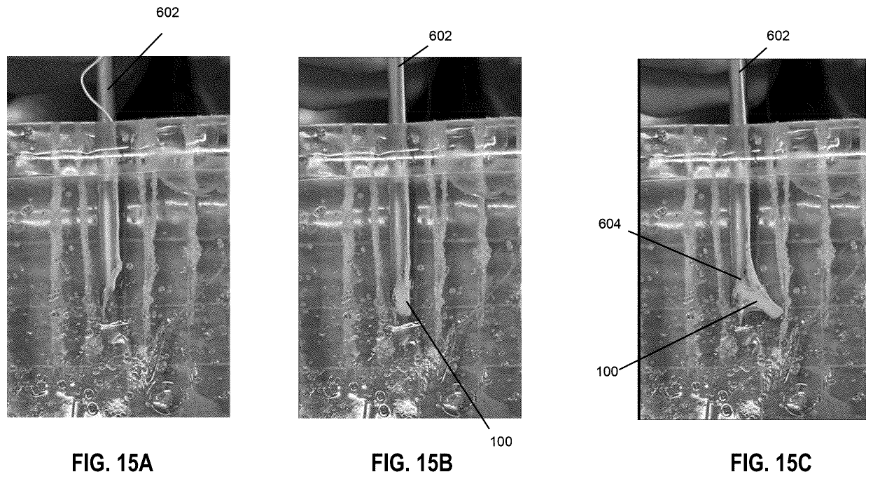

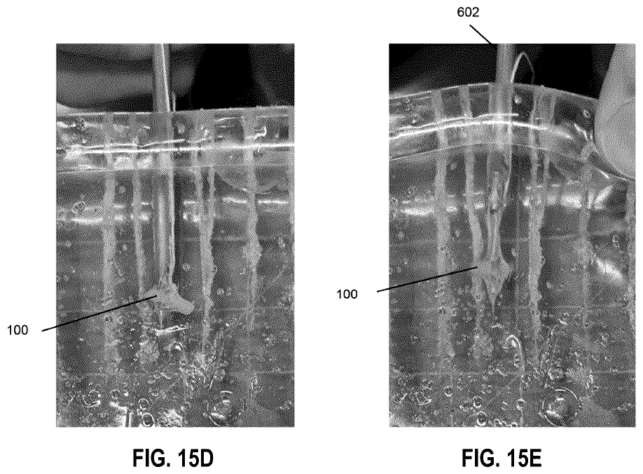

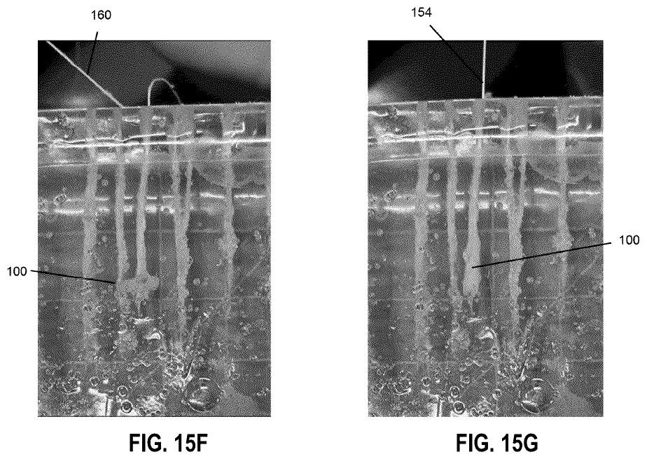

FIGS. 15A-15G illustrate a method of deployment.

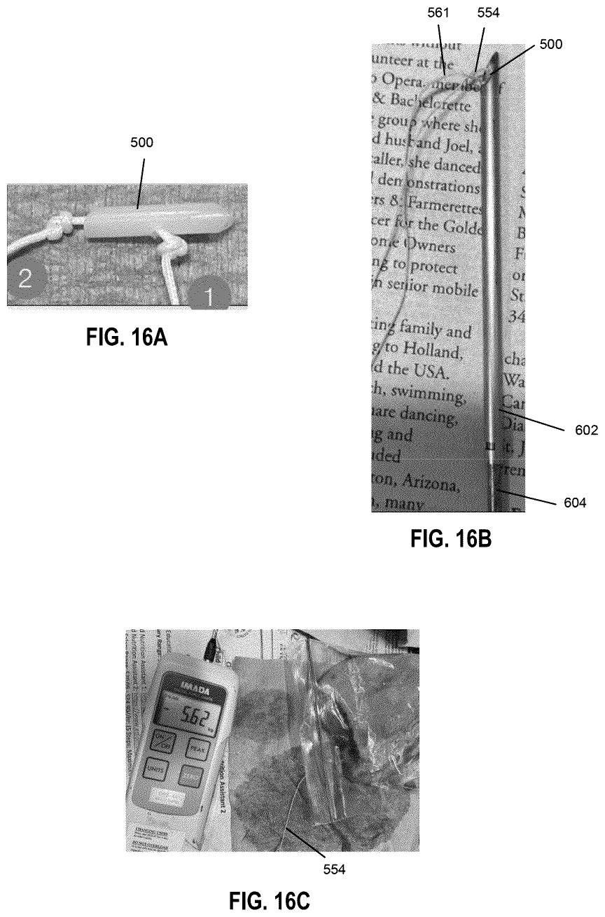

FIGS. 16A-16C illustrate additional features.

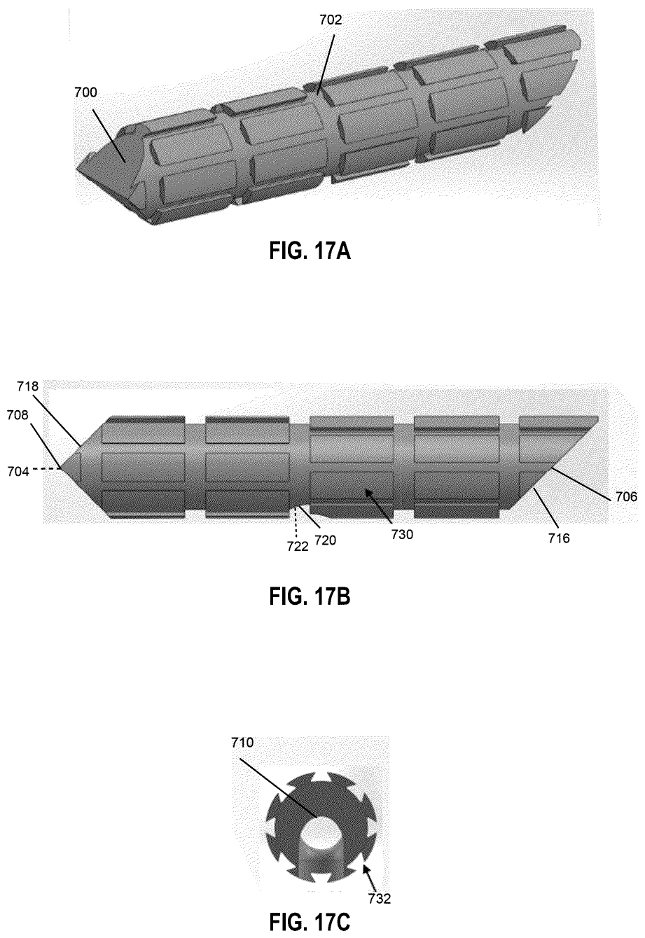

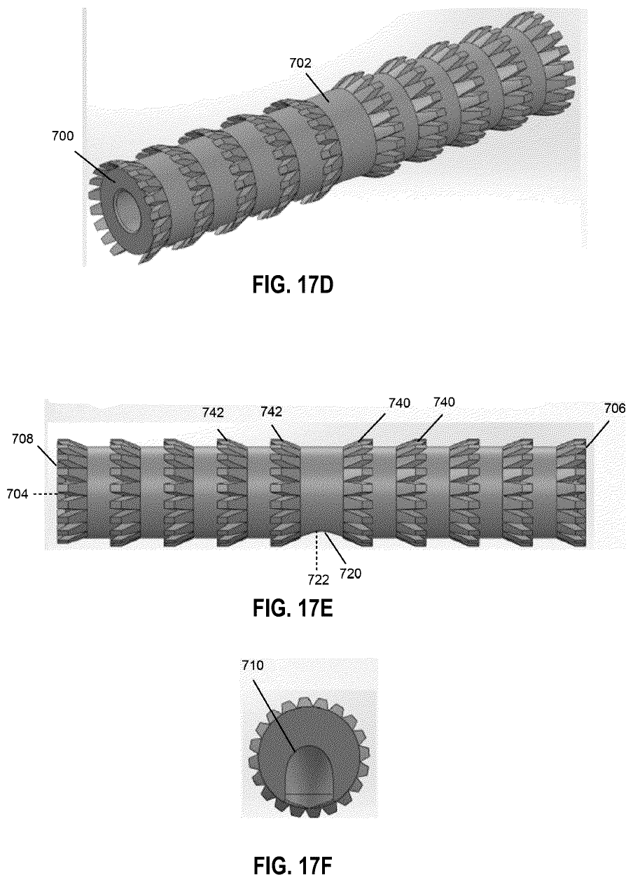

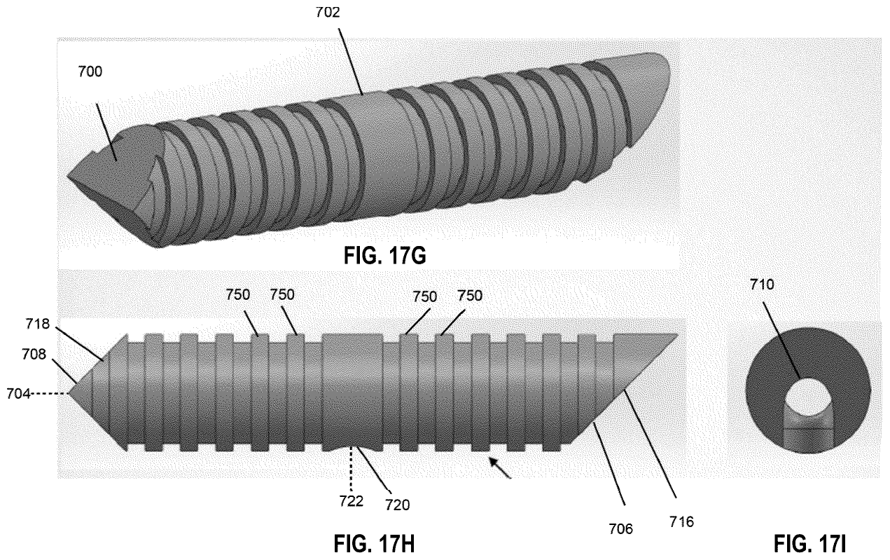

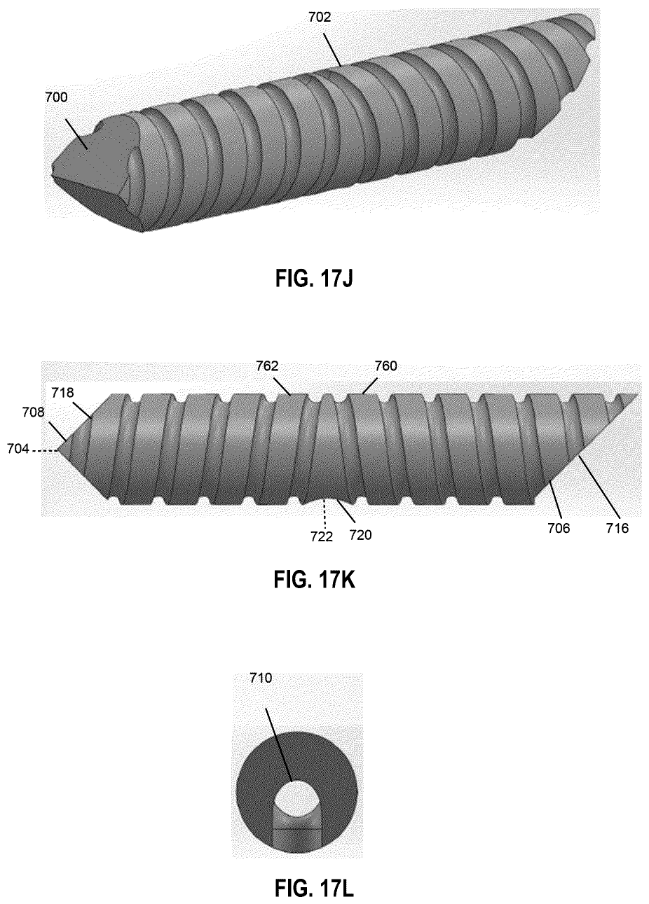

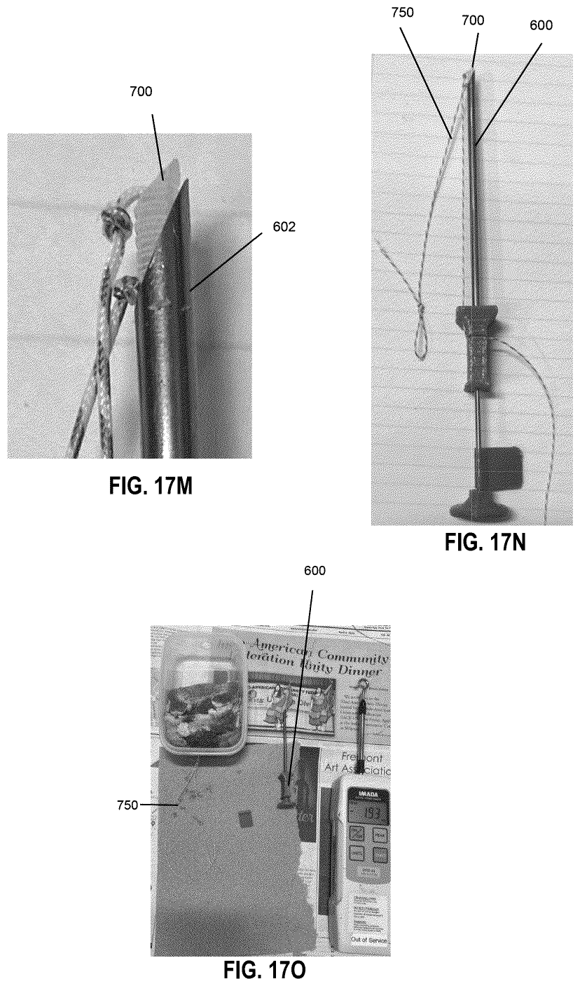

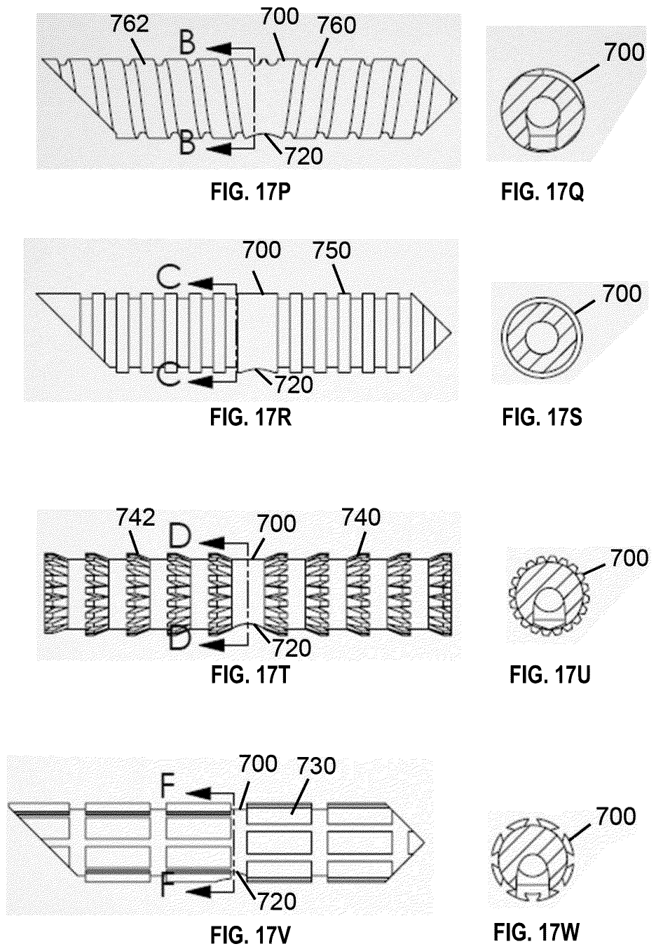

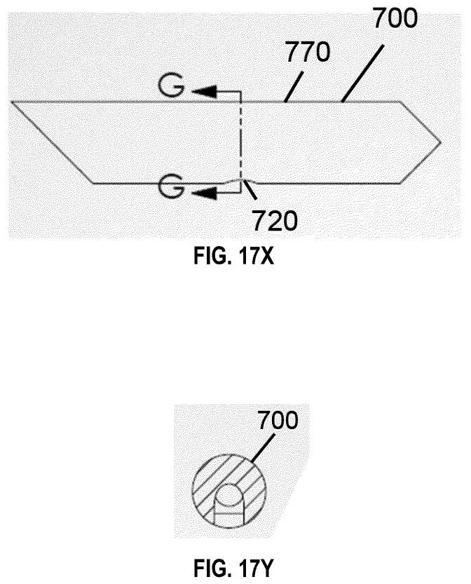

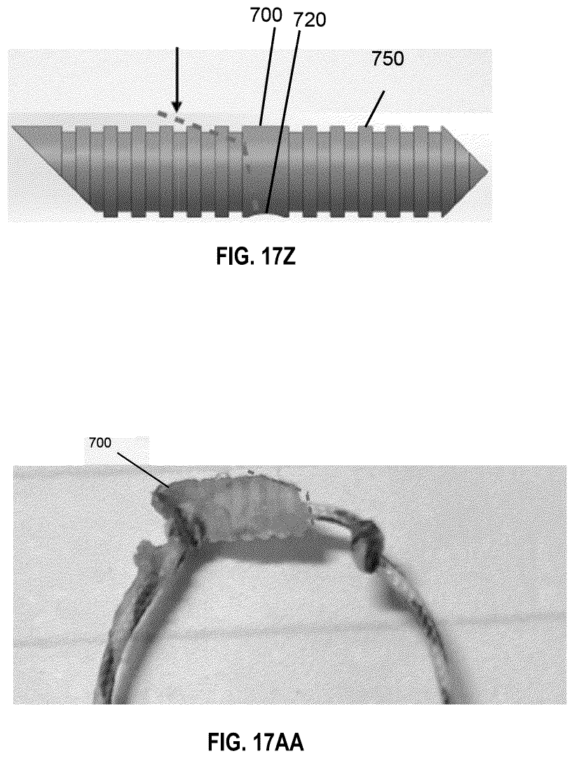

FIGS. 17A-17AA illustrate features of the tissue anchor.

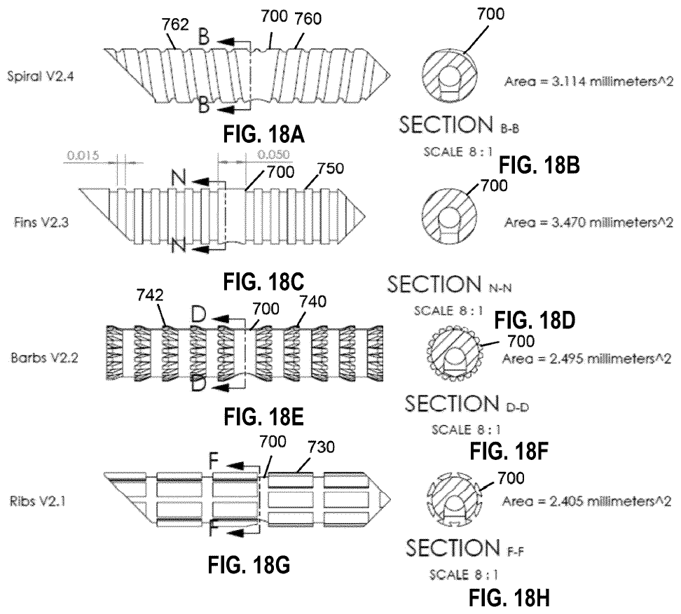

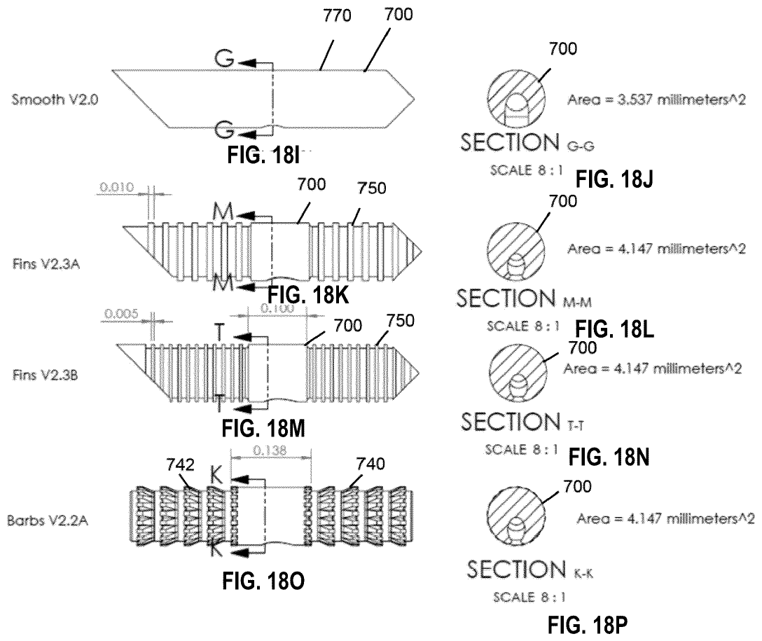

FIGS. 18A-18P illustrate features of the tissue anchor.

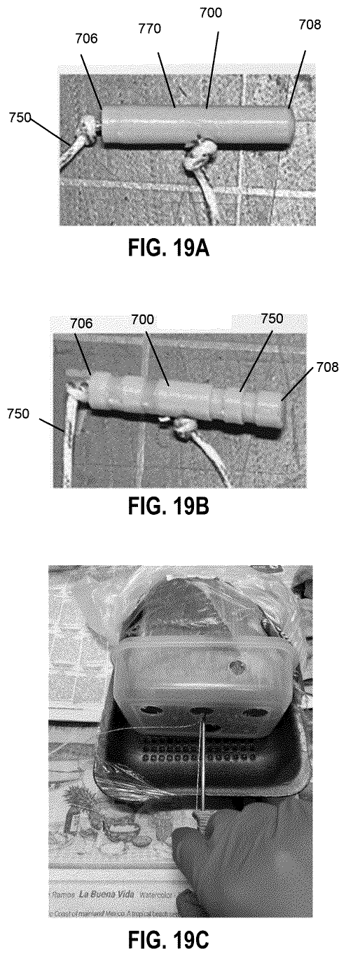

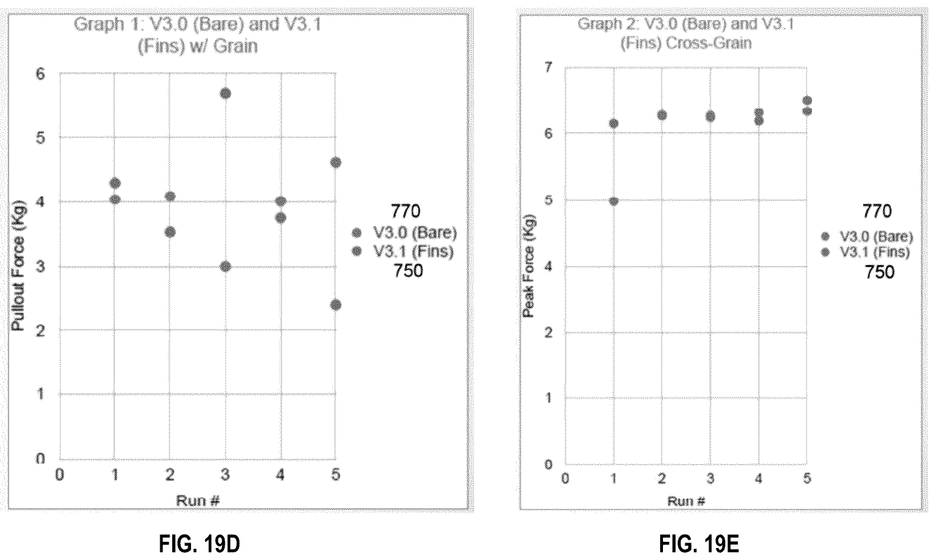

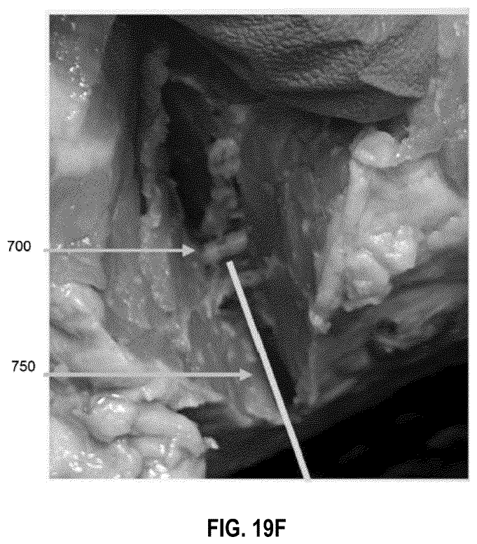

FIGS. 19A-19F illustrate a method of deployment.

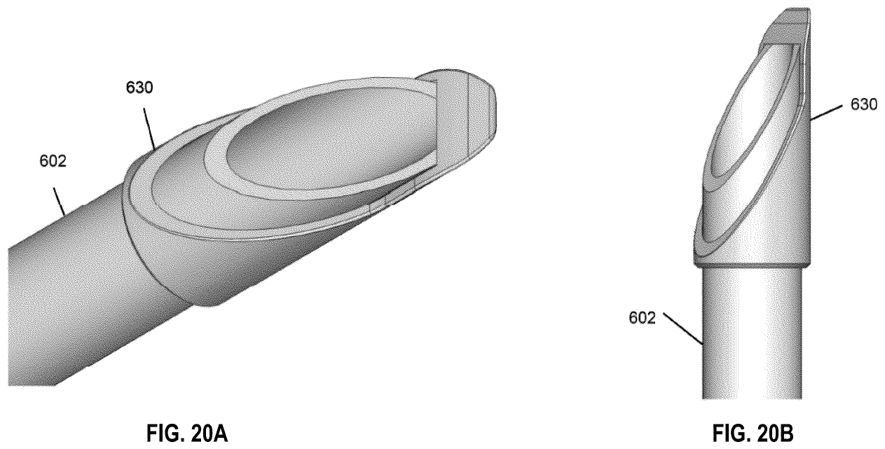

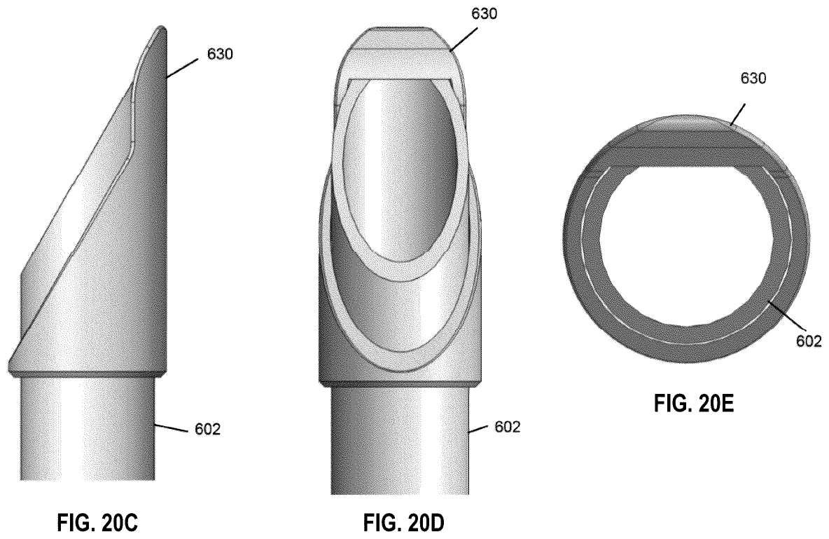

FIG. 20A-20E illustrate a nose cone.

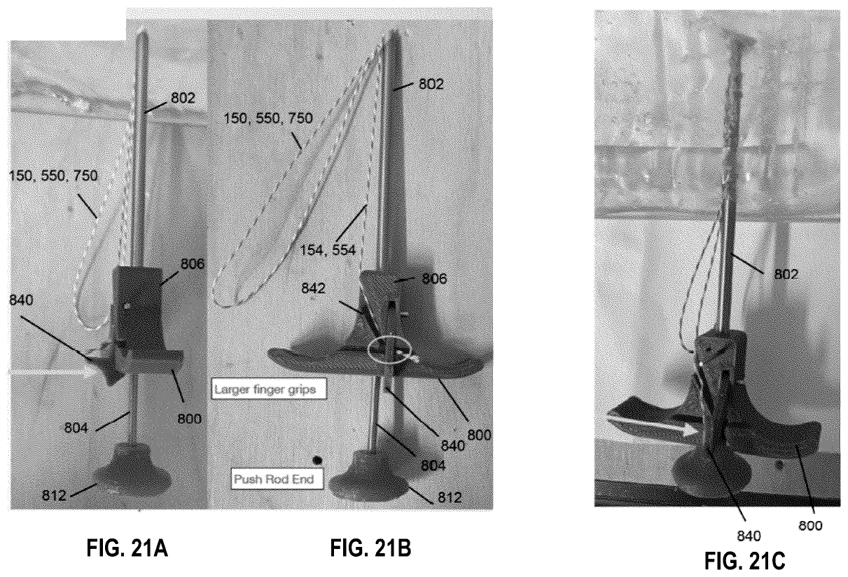

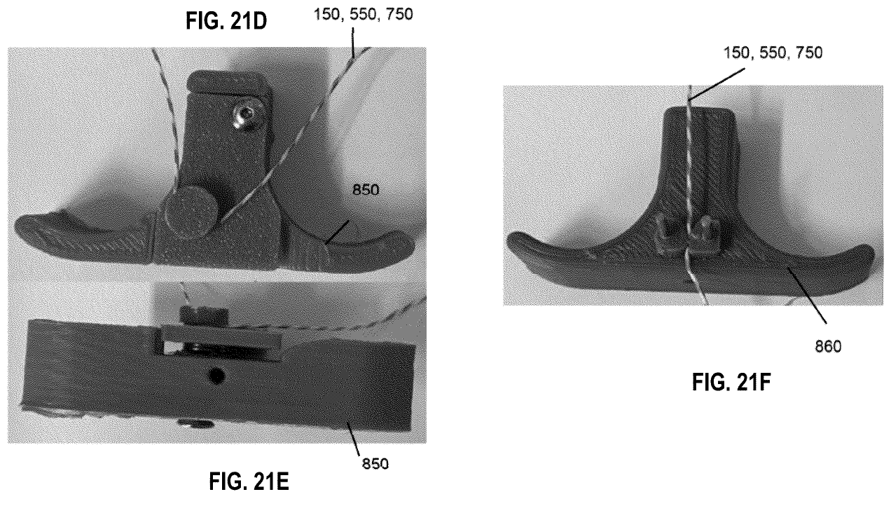

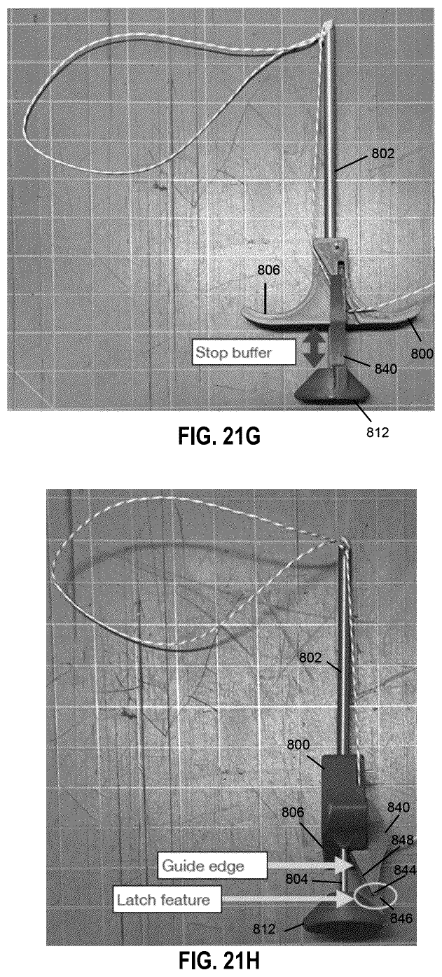

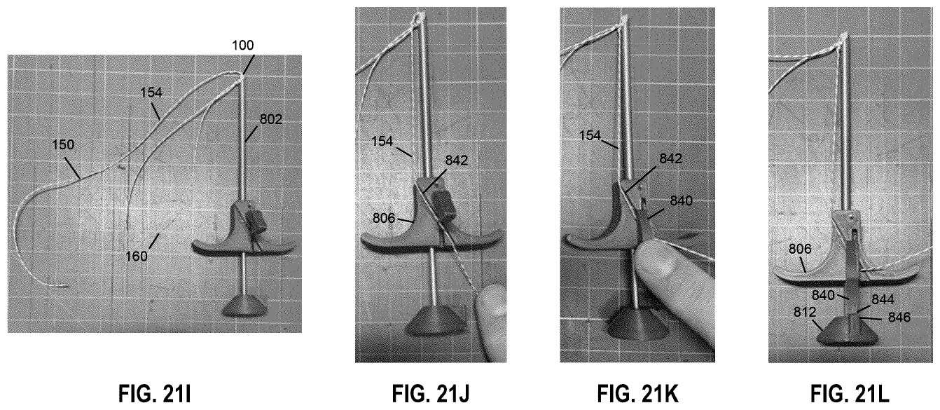

FIGS. 21A-21L illustrate features of the delivery system.

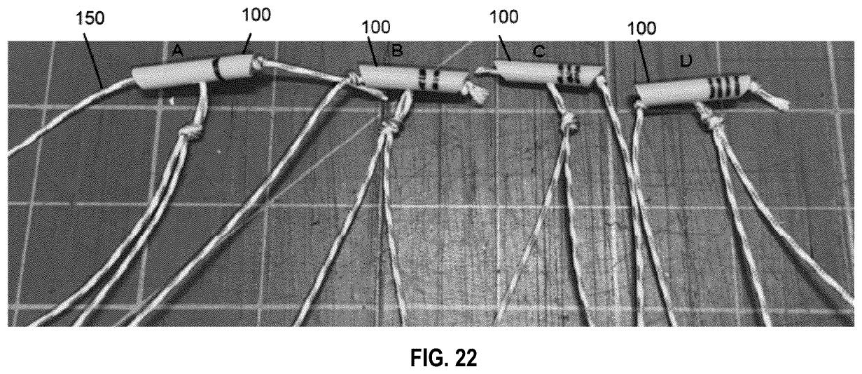

FIG. 22 illustrates multiple tissue anchors.

### DETAILED DESCRIPTION

In some embodiments, disclosed herein are improved systems and methods to suspend tissue in a permanent or temporary way. Disclosed herein are tissue anchors designed to anchor to a point inside tissue. The tissue anchor can be coupled to a suture. To achieve tissue suspension, a suture tail or a suture loop can be tensioned and secured to a bone anchor to achieve tissue suspension. The relative tension between the tissue anchor and the bone anchor can be used for moving or suspending tissue and for maintaining the tissue for an extended period of time. The tissue anchor typically includes an elongated member having a longitudinal axis, with the suture coupled to the elongated member and extending from the elongated member. Disclosed herein are delivery systems that can be used to pass the tissue anchor through tissue and to deploy the tissue anchor within tissue. The suture tail or the suture loop extends through the tissue to be tensioned. As the suture is tensioned, the tissue anchor rotates within the tissue, generally transverse to the suture tail or the suture loop. As the suture is tensioned further, the tissue is moved or suspended. The suture tail or the suture loop is passed through a bone anchor to maintain the position of the tissue. The bone anchor can be anchored to bone, such as the mandible of the patient. In some methods, the tissue anchor assembly is used for the suspension of the genioglossus muscle of the tongue for treating conditions such as obstructive sleep apnea (OSA). The tissue anchor assembly can be used to prevent the tongue from falling back in an anterior-posterior direction. The tissue anchor assembly can be used to prevent the tongue from falling back to contact with the soft palate and posterior pharyngeal wall. The tissue anchor assembly can be used to prevent the tongue from occluding the lower pharyngeal airway in the hypopharynx.

FIGS. 1 and 2 illustrate a tissue anchor assembly 10. The tissue anchor assembly 10 can include a tissue anchor 100. The tissue anchor assembly 10 can include a suture 150. The suture 150 can be coupled to the tissue anchor 100 as described herein. The suture 150 can include green and white threads. The suture 150 can be co-braided polyester fiber. The suture 150 can be nonabsorbable. The suture 150 can be size 2. The tissue anchor assembly 10 can form a unity structure for implanting with tissue. FIGS. 3A-3D illustrate views of the tissue anchor 100. FIG. 4 illustrates an enlarged view of the tissue anchor assembly 10. FIG. 5 illustrates a schematic view of the tissue anchor assembly 10.

The tissue anchor 100 can include an elongated member 102. The elongated member 102 can include a longitudinal axis 104. The longitudinal axis 104 can extend from a first end 106 to a second end 108. The elongated member 102 can include any elongated shape including cylinder, cuboid, triangular prism, hexagonal prism, truncated cone, or any other elongate shape. The elongated member 102 can include a lumen 110. The lumen 110 can extend along the longitudinal axis 104. The lumen 110 can extend the entire length of the elongated member 102. The lumen 110 can include a diameter or cross-sectional dimension. The diameter of the lumen 110 can be 0.15 mm, 0.20 mm, 0.25 mm, 0.30 mm, 0.35 mm, 0.40 mm, 0.45 mm, 0.50 mm, 0.55 mm, 0.60 mm, 0.65 mm, 0.70 mm, 0.75 mm, 0.80 mm, 0.85 mm, 0.90 mm, 0.95 mm, 1.00 mm, 1.05 mm, 1.10 mm, 1.15 mm, 1.20 mm, 1.25 mm, 1.30 mm, 1.35 mm, 1.40 mm, 1.45 mm, 1.50 mm, or any range of two of the foregoing values. The elongated member 102 can include a diameter or cross-sectional dimension. The diameter of the elongated member 102 can be 1.00 mm, 1.10 mm, 1.20 mm, 1.30 mm, 1.40 mm, 1.50 mm, 1.60 mm, 1.70 mm, 1.80 mm, 1.90 mm, 2.00 mm, 2.10 mm, 2.20 mm, 2.30 mm, 2.40 mm, 2.50 mm, 2.60 mm, 2.70 mm, 2.80 mm, 2.90 mm, 3.00 mm, 3.10 mm, 3.20 mm, 3.30 mm, 3.40 mm, 3.50 mm, 3.60 mm, 3.70 mm, 3.80 mm, 3.90 mm, 4.00 mm, 4.50 mm, 5.00 mm, 5.50 mm, 6.00 mm, 7.00 mm, or any range of two of the foregoing values.

The elongated member 102 can include one or more tapered edges. The first end 106 can include a beveled edge 116. The second end 108 can include a beveled edge 118. The beveled edge 116 can slant downward and inward. The beveled edge 118 can slant downward and inward. The beveled edge 116 and the beveled edge 118 can taper inward. The first end 106 can be blunt. The first end 106 can be sharpened. The second end 108 can be blunt. The second end 108 can be sharpened. The tissue anchor 100 can include one beveled edge. The tissue anchor 100 can include two beveled edges. The beveled edges 116, 118 can optimize rotation into a locked position of the elongated member 102 perpendicular to the suture 150. In some embodiments, the first end 106 can be straight, non-beveled, or rounded. In some embodiments, the second end 108 can be straight, non-beveled, or rounded.

The elongated member 102 can include an opening 120. The opening 120 can extend from the outer surface of the elongated member 102 to the lumen 110. The opening 120 can be located along the length of the elongated member 102. The opening 120 can be located at a midpoint of the elongated member 102. The opening 120 can be located at the midpoint to equally distribute forces between the first end 106 and the second end 108. The opening 120 can be located closer to the first end 106 than the second end 108. The opening 120 can be located closer to the second end 108 than the first end 106. The opening 120 can include a through axis 122. The through axis 122 can be transverse to the longitudinal axis 104. The opening 120 can be perpendicular to the lumen 110.

The suture 150 can have a diameter or cross-sectional dimension. The diameter of the lumen 110 can be sized to be greater than the diameter of the suture 150. The diameter of the suture 150 can be 0.05 mm, 0.10 mm, 0.15 mm, 0.20 mm, 0.25 mm, 0.30 mm, 0.35 mm, 0.40 mm, 0.45 mm, 0.50 mm, 0.55 mm, 0.60 mm, 0.65 mm, 0.70 mm, or any range of two of the foregoing values. In some embodiments, the suture 150 is a USP size 2 suture having a diameter of between 0.50 mm and 0.599 mm. In some embodiments, the diameter of the lumen 110 is 1.00 mm. The diameter of the suture 150 can be approximately half the diameter of the lumen 110 of the elongated member 102. The suture 150 can include colored fibers, such as green and white as illustrated. The suture 150 can include a co-braided polyester fiber. The suture 150 can nonabsorbable. The suture 150 can be USP size 3 (diameter between 0.600 mm and 0.699 mm), USP size 2 (diameter between 0.500 mm and 0.599 mm), USP size 1 (diameter between 0.400 mm and 0.499 mm), USP size 0 (diameter between 0.350 mm and 0.399 mm), USP size 2-0 (diameter between 0.300 mm and 0.339 mm), USP size 3-0 (diameter between 0.200 mm and 0.249 mm), USP size 4-0 (diameter between 0.150 mm and 0.199 mm), USP size 5-0 (diameter between 0.100 mm and 0.149 mm), USP size 6-0 (diameter between 0.070 mm and 0.099 mm), USP size 7-0 (diameter between 0.050 mm and 0.069 mm), USP size 8-0 (diameter between 0.040 mm and 0.049 mm), or USP size 9-0 (diameter between 0.030 mm and 0.039 mm), or any range of two of the foregoing values.

The suture 150 can be threaded through the tissue anchor 100. The suture 150 and the tissue anchor 100 are separate components configured to be coupled together. The suture 150 can include a first end 152. The first end 152 of the suture 150 can extend from the first end 106 of the elongated member 102. The suture 150 can include a suture tail 154. The suture tail 154 can extend from the first end 152 of the suture 150 to the first end 106 of the elongated member 102. In some embodiments, the length of the suture tail 154 can be about 101 mm, or 4 inches. The suture 150 can include a first end knot 156. The first end knot 156 can have a diameter. The diameter of the first end knot 156 of the suture 150 is greater than the diameter of the lumen 110 of the elongated member 102. The first end knot 156 can be formed by any knot tying technique including an overhand knot, a figure eight knot, Ashley stopper, or double overhand stopper knot.

The first end knot 156 can be positioned near the first end 106 of the elongated member 102. The first end knot 156 can be positioned against and abut the first end 106 of the elongated member 102. The first end knot 156 can be positioned at the lumen 110 of the elongated member 102. The suture 150 can be threaded through the elongated member 102. The suture 150 can be inserted into the lumen 110 of the elongated member 102. The diameter of the suture 150 is less than the diameter of the lumen 110 of the elongated member 102. The suture 150 can be inserted into the opening 120 of the elongated member 102. The suture 150 can form a suture loop 160 through the opening 120 of the elongated member 102. The suture loop 160 can extend from the opening 120 and back to the opening 120. In some embodiments, the length of the suture loop 160 can be about 101 mm, or 4 inches. In some embodiments, the length of the suture loop 160 is approximately equal to the length of the suture tail 154. The suture loop 160 can include a suture loop knot 162. The suture loop knot 162 can be formed by any knot tying technique including an overhand knot, a figure eight knot, Ashley stopper, or double overhand stopper knot. The suture loop knot 162 can be positioned near the opening 120. The suture loop knot 162 can be spaced apart from the opening 120. The suture 150 can be further threaded through the tissue anchor 100. The suture 150 can be inserted into the lumen 110 of the elongated member 102.

The suture 150 can include a second end 164. The second end 164 of the suture 150 can extend from the second end 108 of the elongated member 102. The second end 164 can include a second end knot 166. The second end knot 166 can have a diameter. The diameter of the second end knot 166 is greater than the diameter of the lumen 110 of the elongated member 102. The second end 164 of the suture 150 can be shorter than the first end 152 of the suture 150. The second end 164 of the suture 150 can have a short section extending from the second end knot 166. The second end knot 166 can be formed by any knot tying technique including an overhand knot, a figure eight knot, Ashley stopper, or double overhand stopper knot. The first end knot 156 and the second end knot 166 can be the same knot or different knots.

In some embodiments, disclosed herein are improved systems and methods that allow for removal of a placed tissue anchor 100. The tissue anchor 100 can be delivered with the first end 106 as the leading end. The suture 150 can include the suture tail 154. The suture tail 154 can be manipulated by the user. The suture tail 154 acts as the release suture if the tissue anchor assembly 10 ever needs to be removed. This tissue anchor assembly 10 uses the continuous suture 150 that, from left to right of the figures, has a suture tail 154 that acts as the release suture if the tissue anchor 100 ever needs to be removed, runs in the suture loop 160 to provide the tether to the bone anchor which can be implanted on the mandible, and exits out the right side of the elongated body 102 where the suture 150 is terminated with the second end knot 166 and cut.

In some embodiments, the suture tail 154 of the suture 150 facilitates positioning of the tissue anchor 100 within tissue. The tissue anchor 100 can be delivered with the first end 106 being the leading end. The tissue anchor 100 can have a low profile insertion configuration. The tissue anchor 100 can be aligned with the insertion direction. The tissue anchor 100 can rotate after being delivered. The tissue anchor 100 can rotate by applying tension to the first end 152. The tissue anchor 100 can rotate by applying tension to the suture tail 154. The tissue anchor 100 can rotate to engage tissue with the beveled edges 116, 118. The beveled edges 116, 118 can optimize rotation into a locked position perpendicular to the suture loop 160.

In some embodiments, the suture tail 154 of the suture 150 facilitates removal of the tissue anchor 100. The tissue anchor 100 can be positioned within tissue. The tissue anchor 100 can be generally perpendicular to the suture loop 160. The tissue anchor 100 can rotate for removal. The tissue anchor 100 can rotate by applying tension to the first end 152. The tissue anchor 100 can rotate by applying tension to the suture tail 154. The tissue anchor 100 can rotate to be parallel to the suture loop 160. The tissue anchor 100 can have a low profile removal configuration. The tissue anchor 100 can be aligned with the removal direction. The tissue anchor 100 can be removed by applying tension to the first end 152 and/or the suture loop 160 once the tissue anchor 100 is rotated.

The suture loop 160 can centrally placed relative to the tissue anchor 100. The suture tail 154 of the suture 150 can extend from one end of the tissue anchor 100. The suture 150 is relatively fixed relative to the tissue anchor 100. When placed in tissue, pulling on the suture tail 154 will lock the tissue anchor 100 by rotating the tissue anchor 100. The tissue anchor 100 is rotated from generally vertical to generally horizontal. The tissue anchor 100 can be perpendicular to the suture loop 160 when locked. Further pulling on the suture tail 154 can allow the tissue anchor 100 to align with the suture loop 160. The tissue anchor 100 is rotated from generally horizontal to generally vertical. The tissue anchor 100 can be repositioned to a different orientation within the tissue by pulling the suture tail 154. The tissue anchor 100 can rotate counterclockwise when the suture tail 154 is tensioned. The tissue anchor 100 can be repositioned within tissue The tissue anchor 100 can be removed.

Referring to FIGS. 3A-3D, an example of the dimensions of the tissue anchor 100, in inches, is illustrated. The tissue anchor 100 can have a length of 0.510 inches, or 12.95 mm. The first end 106 and the second end 108 can have the same taper. The beveled edge 116 can form an angel of 60 degrees. The beveled edge 118 can form an angel of 60 degrees. The opening 120 can be located at a midpoint at a length of 0.255 inches or 6.48 mm. The opening 120 can have a diameter of 0.055 or 1.4 mm. The diameter of the lumen 110 can have a diameter of 0.040 or 1.02 mm. The diameter of the lumen 110 can be less than the diameter of the opening 120. The opening 120 can extend to the lumen 110. The lumen 110 can extend the entire length of the tissue anchor 100. The diameter of the tissue anchor 100 can be 0.11 inches or 2.79 mm. The tissue anchor 100 can be formed form a polymer such as PEEK.

Referring to FIG. 5, the first end knot 156 and the second end knot 166 couple the suture 150 to the tissue anchor 100 along the longitudinal axis 104. The suture loop knot 162 couples the suture 150 to the tissue anchor 100 along the through axis 122. The knots 156, 162, 166 couple the suture 150 to the tissue anchor 100 at three points. The knots 156, 162, 166 couple the suture 150 to the tissue anchor 100 along two transverse axes. In some embodiments, the suture 150 has little to no suture stretch under normal anatomic loads. In some embodiments, the suture 150 has slight suture stretch with loads up to 8.2 kg. In some embodiments, the suture 150 has no signs of breakage or knots slipping.

The first end knot 156 can be an overhand knot. The first end knot 156 can be positioned near or adjacent to the lumen 110 of the elongated member 102. The second end knot 166 can be an overhand knot. The second end knot 166 can be positioned near or adjacent to the lumen 110 of the elongated member 102. The suture loop knot 162 can be an overhand knot. The suture loop knot 162 can be spaced apart from the elongated member 102 by a distance of 0.13 inches to 0.18 inches, or 3.3 mm to 4.8 mm.

FIGS. 6A-6E are view of an embodiment of the tissue anchor 100 and the suture 150 and a bone anchor 200. The tissue anchor assembly 10 can be tested for fatigue. In some embodiments, sutures may fail within five to ten million cycles. The tissue anchor assembly 10 was designed to improve fatigue. FIG. 6A is a fatigue tester 300. The fatigue tester 300 can include a cycle counter 302. The fatigue tester 300 can include a cycle counter finger 304. The fatigue tester 300 can include a cam pivot point 306. The fatigue tester 300 can include a counter sensor 308. The fatigue tester 300 can include a range adjustment slide 310. The fatigue tester 300 can include a fixed tie rod 312. The fatigue tester 300 can include a linear slide 314. The fatigue tester 300 can include a compression spring 316. The fatigue tester 300 can include a load support block 318. The fatigue tester 300 can include a tissue anchor support block 320. The fatigue tester 300 can include a bone anchor mount 322. The tissue anchor 100 can be coupled to the tissue anchor support block 320. The bone anchor 200 can be coupled to the bone anchor mount 322. The suture 150 can extend from the tissue anchor 100 to the bone anchor 200. The fatigue tester 300 can apply a force to the tissue anchor 100 to replicate the forces exerted by a patient's anatomy. The cycle counter finger 304 can rotate relative to the counter sensor 308 to track the number of cycles. The cam pivot point 306 rotates causing the fixed tie rod 312 to compress the compression spring 316. This motion of the fixed tie rod 312 causes corresponding forces to be applied to the tissue anchor 100. FIG. 6B is a side view. The fatigue tester 300 can include a motor 324. The motor 324 rotates the cycle counter finger 304. The fixed tie rod 312 rotates as well, and slides along the range adjustment slide 310. The motion of the fixed tie rod 312 exerts a cyclic force on the tissue anchor 100.

FIG. 6C is a view of the tissue anchor 100 and the suture 150 after ten million cycles. The suture loop 160 exhibited zero signs of wear. Embodiments of the suture with a single suture extending through the opening, as described herein, exhibit fatigue at 6.8 million cycles wherein the single suture frayed and broke near the opening 120.

FIGS. 6D and 6E are views of the suture 150 and the bone anchor 200. The suture 150 exits the bone anchor 200 in FIG. 6D. The suture 150 enters the bone anchor in FIG. 6E. The black marks are used to visualize the suture stretch and/or slippage. The marks remained in place during the ten million cycles. The suture 150 did not experience stretch and/or slippage.

The fatigue tester 300 was run for over ten million cycles. The cycles were 7.58 Hz, thereby achieving 650 k cycles per day. The loading force range was set to cycle between 0.5 kg and 4.1 kg. In some embodiments, the suture 150 required a conditioning period to compensate for stretch and knot tightening. For instance, at 800,000 cycles, the suture 150 stretched and the maximum load dropped from 4.1 kg to 3.9 kg. In some embodiments, retightening the suture 150 in the bone anchor 200 allowed for the load to be back to the 4.1 kg peak for the remainder of the ten million cycles without any signs of slippage, stretch, or fraying. The final load range was 0.6 kg to 3.9 kg. The tissue anchor 100 was able to withstand ten million cycles to 4 kg without signs of wear on the material of the tissue anchor or the suture material.

FIGS. 7A-7B are view of an embodiment of the bone anchor 200. The bone anchor 200 can include a proximal head portion 202 and a distal threaded portion 204. The bone anchor 200 can have a suture locking and tension mechanism such as a locking screw 210. The locking screw 210 can be within an eyelet 211 of the bone anchor 200. The eyelet 211 can extend completely through two sides of the proximal head portion 202 of the bone anchor and have a longitudinal axis that is transverse to the longitudinal axis of the bone anchor 200. In some embodiments, the locking screw 210 has an enlarged proximal head portion 222 having an opening on its upper surface, e.g., a hex configuration that may be used to engage a screwdriver or other instrument. The locking screw 210 has a drive portion 224 configured to rotate and threadably engage an internally threaded surface 220 extending from the upper surface of the proximal head portion 202 and in communication with the eyelet 211. Appropriate rotation of the locking screw 210 with respect to the internally threaded surface 220 will decrease the available space within the eyelet 211 such that the suture 150 is reversibly trapped within the eyelet 211 by the locking screw 210. The eyelet 211 defines a cavity within the bone anchor 200 that the locking screw 210 may not entirely fill. Rather, between a distal surface 214 of the locking screw 210 and a distal surface 216 of the eyelet 211 defines a gap 212. The gap 212 is sufficiently small that the suture 150 remains fixed within the eyelet 211 while the locking screw 210 is appropriately secured within the eyelet 211 (e.g., via complementary threaded surfaces of the locking screw 210 and eyelet 211 as noted above), however, the gap 212 advantageously helps to minimize damage to the suture 150. In other words, the height (a distance measured parallel to the longitudinal axis of the bone anchor 200) of the gap 212 should be sized and configured to adequately secure the suture 150 while avoiding over-compression of the locking screw 210 to the suture 150. In some embodiments, the gap 212 or height dimension is between about 0.005 inches and 0.02 inches, such as about 0.01 inches. When the height of the gap is configured to adequately secure the suture 150 without over-compressing, a desired minimum pull-out force for the suture 150 can be in some embodiments about 4 kgf to about 8 kgf, or more preferably about 5 kgf to about 7 kgf, or at least about 4 kgf, 5 kgf, 6 kgf, 7 kgf, 8 kgf, or more in some embodiments. In some embodiments, the gap 212 is 0.008 inches or 0.20 mm.

FIG. 8 illustrates an embodiment of the tissue anchor assembly 10 in relation to a mandible 400. The tissue anchor 100 can be positioned in the tongue operably coupled to the suture 150, which in turn can be connected to the bone anchor 200 anchored to the mandible 400. The suture 150 can move under load to accommodate swallowing and speech. The tissue anchor 100, the suture 150, and the bone anchor 200 are entirely within the tissue. The bone anchor 200 can be anchored to the mandible 400. The suture loop 160 can be secured within the gap 212 of the bone anchor 200. In some embodiments, the suture tail 154 also secured within the gap 212 of the bone anchor 200. In some embodiments, the suture tail 154 is not secured to the bone anchor 200. The suture tail 154 can be used to remove the tissue anchor 100, if needed. The suture loop 160 provides a site to anchor to the bone anchor 200. The suture loop 160 can be tensioned to adjust the position of the tissue, such as tissue of the tongue. FIG. 8 is a sketch of the tissue anchor assembly 10 in its intended use for tongue suspension. This view is from the side with tether lines running to the mandible 400.

FIG. 9 illustrates an embodiment of the tissue anchor assembly 10 in relation to the mandible 400. The tissue anchor 100 can be coupled to the suture 150 before use. The knots 156, 166, 162 can be tied before use. The tissue anchor 100 can be inserted into tissue with the suture 150 coupled thereto. The tissue anchor 100 can rotate within tissue to a locked position. The suture 150 can extend from the tissue anchor 100. The suture 150, or a portion thereof, such as the suture loop 160 can be coupled to the bone anchor 200. The suture loop 160 can be tensioned before coupling with the bone anchor 200 to adjust the position of the tissue, such as tissue of the tongue. The bone anchor 200 can be anchored to the mandible 400. The tissue anchor 100 and the suture 150 can move under normal anatomical loads. The tissue anchor 100, the suture 150, and the bone anchor 200 are entirely within the patient. FIG. 9 is a sketch of the tissue anchor assembly 10 in its intended use for tongue suspension. This view is looking down at the tongue base with the implant being delivered to the posterior portion of the tongue base.

The design of the tissue anchor assembly 10 uses the continuous suture 150 that has the suture tail 154 that exits out the first end 106 of the tissue anchor 100 that acts as the release suture if the tissue anchor 100 ever need to be removed. The continuous suture 150 has the first end 152 and the suture tail 154. The continuous suture 150 has the first end knot 156. The continuous suture 150 runs through the tissue anchor 100 and exits at the opening 120. The continuous suture 150 runs in the suture loop 160 to allow the suture loop 160 to be anchored to the bone anchor 200, which can be implanted to bone such as on the mandible. The continuous suture 150 has the suture loop knot 162. The continuous suture 150 exits out the second end 108 of the tissue anchor 100 where the suture 150 is terminated with the second end knot 166 and cut. The continuous suture 150 has the second end knot 166. The continuous suture 150 extends from the first end 152 to the second end 164.

The design of the tissue anchor assembly 10 can have several advantages. The design balances the tether tension equally to each end of the tissue anchor 100. The opening 120 can be located at a midpoint of the tissue anchor 100. The tension applied to the suture loop 160 can apply equal tension to the first end 106 and the second end 108 of the tissue anchor 100. The suture loop 160 can be tensioned before anchoring to the bone anchor 200. The tension on the suture loop 160 can move or lift tissue, such as tissue of the tongue. The tension on the suture loop 160 can suspend the genioglossus muscle of the tongue to treat conditions such as obstructive sleep apnea (OSA). The tension of the suture loop 160 can be adjusted. The bone anchor 200 can allow for adjustment of the suture loop 160 by loosening the locking screw 210, adjusting the tension of the suture loop 160, and tightening the locking screw 210. The suture loop 160 can pass through the gap 212 in the bone anchor 200 when the suture loop 160 is secured.

The design minimizes wear of the tissue anchor 100 and the suture 150. The design includes overhand knots 156, 162, 166. The knots 156, 162, 166 are integrally formed with the suture 150. The knots 156, 162, 166 provide robust coupling between the tissue anchor 100 and the suture 150. The knots 156, 162, 166 have the same material properties of the suture 150. In some embodiments, the design does not require additional adhesives for anchoring. In some embodiments, the design does not require adhesives which may wear over time. The tissue anchor 100 can be formed of robust material such as PEEK. The suture 150 can be formed of robust materials such as co-braided polyester fibers. The design advantageously shows little wear over 10 million cycles. The design advantageously shows little fatigue or failure.

The design maintains the profile of the tissue anchor 100 with the suture 150 only going all the way through the tissue anchor 100 via the axial center opening 120. The tissue anchor 100 has a sleek cylindrical profile for delivery. The tissue anchor 100 can be easily loaded into the delivery system. The tissue anchor 100 can be inserted into tissue along the longitudinal axis of the delivery system. The tissue anchor assembly 10 includes the suture 150. The suture 150 can be threaded through the tissue anchor 100 to maintain a low profile for delivery. The suture loop 160 and the suture tail 154 exit the tissue anchor 100. The suture loop 160 exits the tissue anchor via the opening 120. The suture loop 160 can maintain a low profile for delivery. The suture tail 154 exits the tissue anchor via the first end 106. The suture tail 154 can maintain a low profile for delivery.

FIG. 10 illustrates a view of a tissue anchor assembly 50. The tissue anchor assembly 50 can include any of the features or methods of use of the tissue anchor assembly 10 described herein. The tissue anchor assembly 50 can include a tissue anchor 500. The tissue anchor assembly 50 can include a suture 550. The tissue anchor 500 can include an elongated member 502. The elongated member 502 can include a longitudinal axis 504. The longitudinal axis 504 can extend from a first end 506 to a second end 508. The elongated member 502 can include any elongated shape. The elongated member 502 can include a lumen 510. The lumen 510 can extend along the longitudinal axis 504. The lumen 510 can extend the entire length of the elongated member 502. The lumen 510 can extend a portion of the length of the elongated member 502, such as from the first end 506 to a midpoint along the length of the elongated member 502. The lumen 510 can include a diameter or cross-sectional dimension. The diameter of the lumen 510 can be 0.15 mm, 0.20 mm, 0.25 mm, 0.30 mm, 0.35 mm, 0.40 mm, 0.45 mm, 0.50 mm, 0.55 mm, 0.60 mm, 0.65 mm, 0.70 mm, 0.75 mm, 0.80 mm, 0.85 mm, 0.90 mm, 0.95 mm, 1.00 mm, 1.05 mm, 1.10 mm, 1.15 mm, 1.20 mm, 1.25 mm, 1.30 mm, 1.35 mm, 1.40 mm, 1.45 mm, 1.50 mm, or any range of two of the foregoing values.

The elongated member 502 can include one or more rounded edges. The first end 506 can include a tapered edge 516. The second end 508 can include a double tapered edge 518. The tapered edge 516 can slant downward and inward. The double tapered edge 518 can rounded. The first end 506 can be blunt. The first end 506 can be sharpened. The second end 508 can be blunt. The second end 508 can be sharpened.

The elongated member 502 can include an opening 520. The opening 520 can extend from the outer surface of the elongated member 502 to the lumen 510. The opening 520 can be located along the length of the elongated member 502. The opening 520 can be located at a midpoint of the elongated member 502. The opening 520 can be located closer to the first end 506 than the second end 508. The opening 520 can be located closer to the second end 508 than the first end 506. The opening 520 can include a through axis 522. The through axis 522 can be transverse to the longitudinal axis 504. The opening 520 can be perpendicular to the lumen 510. The tissue anchor 500 can be formed form a polymer such as PEEK.

The suture 550 can have a diameter or cross-sectional dimension. The diameter of the lumen 510 can be sized to be greater than the diameter of the suture 550. The diameter of the suture 550 can be 0.05 mm, 0.10 mm, 0.15 mm, 0.20 mm, 0.25 mm, 0.30 mm, 0.35 mm, 0.40 mm, 0.45 mm, 0.50 mm, 0.55 mm, 0.60 mm, 0.65 mm, 0.70 mm, or any range of two of the foregoing values.

The suture 550 can be threaded through the tissue anchor 500. The suture 550 and the tissue anchor 500 are separate components configured to be coupled together. The suture 550 can include a first end 552. The first end 552 can include a first end knot 556. The first end knot 556 can have a diameter. The diameter of the first end knot 556 is greater than the diameter of the lumen 510 of the elongated member 502. The first end knot 556 can be formed by any knot tying technique including an overhand knot, a figure eight knot, Ashley stopper, or double overhand stopper knot.

The first end knot 556 can couple the suture 550 to the tissue anchor 500 along the longitudinal axis 504. The first end knot 556 can be an overhand knot. The first end knot 556 can be positioned near or adjacent to the lumen 510 of the elongated member 502. The first end 552 can include a first suture tail 554. The first suture tail 554 can extend for a length, such as 101 mm or 4 inches, similar to the suture tail 154. The first suture tail 554 acts as the release suture if the tissue anchor 500 ever needs to be removed.

The suture 550 can be inserted into the lumen 510 of the elongated member 502. The diameter of the suture 550 is less than the diameter of the lumen 510 of the elongated member 502. The suture 550 can extend through the opening 520. The suture 550 can include the second suture tail 561. The suture 550 can include a second suture tail knot 562. The second suture tail knot 562 can have a diameter. The diameter of the second suture tail knot 562 is greater than the diameter of the opening 520. The second suture tail knot 562 can be formed by any knot tying technique including an overhand knot, a figure eight knot, Ashley stopper, or double overhand stopper knot. In some embodiments, the length of the second suture tail 561 can be about 101 mm, or 4 inches. The second suture tail knot 562 can be positioned near the opening 520. The second suture tail knot 562 can be spaced apart from the opening 520.

The first suture tail 554 of the suture 550 can extend from the first end 506 of the elongated member 502. The second suture tail 561 can extend from the opening 520 of the elongated member 502. In some embodiments, the length of the first suture tail 554 is approximately equal to the length of the second suture tail 561.

The first end knot 556 and the second suture tail knot 562 can be the same knot or different knots. The first end knot 556 couples the suture 550 to the tissue anchor 500 along the longitudinal axis 504. The second suture tail knot 562 couples the suture 550 to the tissue anchor 500 along the through axis 522. The knots 556, 562 couple the suture 550 to the tissue anchor 500 at two points. The knots 556, 562 couple the suture 550 to the tissue anchor 500 along two transverse axes.

The first end knot 556 can be an overhand knot. The first end knot 556 can be positioned near or adjacent the lumen 510 of the elongated member 502. The second suture tail knot 562 can be an overhand knot. The second suture tail knot 562 can be positioned near or adjacent the opening 520 of the elongated member 502. The tissue anchor 500 and suture 550 can be coupled to the bone anchor 200, similar to embodiments described herein.

FIG. 11A-11B illustrates an embodiment of a delivery system 600. The delivery system 600 is a repeatable delivery system that is easy to load and deploy. The delivery system 600 can be used to deploy tissue anchors for testing pullout forces. The delivery system 600 can be 3D printed. The delivery system 600 can be formed of a polymer material such as PLA. The delivery system 600 can include a hypo tube 602. The hypo tube 602 can have a small diameter such as a 0.120 inch outer diameter or 3.05 mm outer diameter. The delivery system 600 can include a push rod 604. The push rod 604 can be at least partially disposed within the hypo tube 602. The smaller hypo tube 602 and the push rod 604 were used to decrease the track size left behind. The hypo tube 602 can be sized to receive the tissue anchor 100, 500. The longitudinal axis 104, 504 of the tissue anchor 100, 500 can align with the longitudinal axis of the delivery system 600.

The delivery system 600 can include a hypo tube handle 606. The hypo tube handle 606 is coupled to the hypo tube 602. The hypo tube handle 606 can include a suture cleat 608. The suture cleat 608 can receive the suture 150, 550. The suture cleat 608 can receive the suture tail 154, 554. The suture cleat 608 can receive the suture loop 160. The suture cleat 608 can receive the second suture tail 561. The delivery system 600 can include a stop 610. The stop 610 can be disposed on the push rod 604. The delivery system 600 can include a push rod handle 612. The push rod handle 612 is coupled to the push rod 604 to control longitudinal movement of the push rod 604. The stop 610 is disposed between the push rod handle 612 and the hypo tube handle 606. The stop 610 is configured to prevent longitudinal movement of the push rod 604 relative to the hypo tube 602 when the stop 610 is positioned between the push rod handle 612 and the hypo tube handle 606. The stop 610 is removable to allow longitudinal movement of the push rod 604 relative to the hypo tube 602.

FIGS. 12A-12D illustrate a method of deployment using the delivery system 600 of FIG. 11A. The tissue anchor 100, 500 is loaded into the hypo tube 602 of the delivery system 600. The suture 150, 550 is inserted into the suture cleat 608. The hypo tube 602 is inserted at a desired depth in FIG. 12A. The stop 610 is removed in FIG. 12B. The hypo tube handle 606 and the hypo tube 602 remain in place. The push rod handle 612 and the push rod 604 is advanced relative to the handle 606 in FIG. 12C. Advancing the push rod 604 advances the tissue anchor 100, 500 from the hypo tube 602. The suture 150, 550 is removed from the suture cleat 608 in FIG. 12D. The delivery system 600 is easy to use. The delivery system 600 can allow for back-to-back deployments of tissue anchors. The delivery system 600 can allow for tissue anchor delivery that is easy to perform and yields very similar results. There were no pullout forces measured in experimental studies.

The delivery system 600 can be a repeatable and easy to use system. The tissue anchor 100, 500 can be easy to load and deploy. The delivery system 600 can be used for delivery in tissue. The delivery system 600 can be used for tissue testing of pull-out forces. The delivery system 600 can be used for back-to-back deployments of tissue anchors 100, 500. The delivery system 600 can produce similar or identical results. The delivery system 600 is shown with the tissue anchor assembly 50 but the tissue anchor assembly 10 can be used. The delivery system 600 can be 3D printed. The delivery system 600 can comprise PLA. The hypo tube 602 can have a smaller diameter such as 0.120″. The push rod 604 can have a smaller diameter such as 0.090″. The smaller diameter hypo tube 602 can decrease the tract size left behind in the tissue. In bench tests, repeated back-to-back deployments were easy to perform and yielded very similar results.

FIGS. 13A-13G illustrate a method of deployment. The tissue anchor 100 can be deployed in ballistic gelatin in a bench test. The deployment method can include the delivery system 600. FIG. 13A illustrates the tissue anchor 100. FIG. 13B illustrates the tissue anchor 100, the suture tail 154 and the suture loop 160. FIGS. 13C and 13D illustrate the loading position of the tissue anchor 100 relative to the hypo tube 602 of the delivery system 600. FIGS. 13E-13G illustrate deployment steps.

The tissue anchor 100 can include any feature described herein. The tissue anchor 100 yielded promising results when performing peak pullout tests. The bench test demonstrated the deployment method. The bench test allowed visualization of the tissue anchor 100 orientation. The bench test utilized ballistic gelatin which has transparent properties. The tissue anchor 100 was deployed using variations of deployment methods.

The deployment method can include one or more of the following steps. The deployment method can include inserting the loaded hypo tube 602 while the hypo tube 602 and the push rod 604 are held fixed together. The hypo tube 602 can be loaded with the tissue anchor 100. The hypo tube 602 can be inserted to a depth into the ballistic gelatin. FIG. 13E illustrates the hypo tube 602 inserted into the ballistic gelatin.

The deployment method can include advancing the push rod 604 until the tissue anchor 100 was fully deployed. The tissue anchor 100 can be fully deployed when the tissue anchor 100 is just beyond tip of chamfer on the hypo tube 602. The push rod 604 can be retracted slightly to create room for tissue anchor 100 to pivot when the suture 150 is tensioned. In some methods, the tissue anchor 100 can be prevented from pivoting when the push rod 604 is touching the tissue anchor 100. In some methods, if the hypo tube 602 and the push rod 604 are removed completely prior to pivoting the tissue anchor 100, then the tissue anchor 100 remains parallel in the insertion tract even when trying to pull on either suture loop 160 or the suture tail 154 and the tissue anchor 100 eventually pulls out. In some methods, the method can include retracting the hypo tube 602 instead of advancing the push rod 604 beyond the edge of the hypo tube 602 to deploy the tissue anchor 100. FIG. 13F illustrates the fully deployed tissue anchor 100. The push rod 604 can be retracted to allow the tissue anchor 100 to pivot.

The deployment method can include pulling suture tail 154 to cause the tissue anchor 100 to pivot to a perpendicular orientation with respect to the insertion tract. The suture tail 154 can be pulled a controlled distance. In some methods, pulling the suture loop 160 requires a high force and does not pivot the tissue anchor 100. FIG. 13G illustrates the pivoted tissue anchor 100. The suture tail 154 can be pulled to pivot the tissue anchor 100. The tissue anchor 100 can be oriented generally perpendicular to the insertion tract of the delivery system 600.

The deployment method can result in a repeatable tissue anchor deposit at approximately 90° to the insertion tract. The deployment method can include pulling the suture tail 154 to pivot the tissue anchor 100. In the ballistic gelatin, the tissue anchor 100 can reorient itself to the insertion tract once the suture loop 160 is pulled hard enough and the tissue anchor 100 can slide out fairly easily. The method of pulling the suture loop 160 could be an advantageous recovery feature if the tissue anchor 100 behaves similarly in tissue as ballistic gelatin.

FIGS. 14A-14J illustrate features of the delivery system. The delivery system 600 can include any feature described herein. The delivery system 600 can include a ramp feature. The tissue anchor 100 can be deployed utilizing the ramp feature. The ramp feature can be advantageous to avoid complicated delivery systems. The ramp feature can facilitate rotating the tissue anchor 100 by ninety degrees. The tissue anchor 100 can include any feature described herein. FIG. 14A illustrates the tissue anchor 100.

The delivery system 600 can include a nose cone 620. The nose cone 620 can include a ramp 622. The ramp 622 can be located on an inside face. The ramp 622 can be disposed within the hypo tube 602 of the delivery system 600. The nose cone 620 can provide a trocar feature to penetrate tissue easily. The tip 624 of the nose cone 620 can be pointed. The tip 624 of the nose cone 620 can be conical. The tip 624 of the nose cone 620 can include a cutting point. FIG. 14B illustrates the nose cone 620.

FIG. 14C illustrates the hypo tube 602 of the delivery system 600 and the nose cone 620. The hypo tube 602 can be a delivery tube. The hypo tube 602 can incorporate the nose cone 620. The nose cone 620 can be crimped into the end of the hypo tube 602. The hypo tube 602 can include an opening 626. The opening 626 can be along the side of the hypo tube 602. The opening 626 of the hypo tube 602 can be lengthened to allow the tissue anchor 100 to articulate through an arc. The nose cone 620 can be coupled to the hypo tube 602 through adhesive, welding, one or more fasteners, one or more rivets, clinching, brazing, or other joining means. The nose cone 620 and the hypo tube 602 can be integrally or monolithically formed.

FIGS. 14D-14H illustrates the deployment of the tissue anchor 100. The tissue anchor 100 can be modified to add a small chamfer 130. The small chamfer 130 can be located on the leading edge of the tissue anchor 100. The small chamfer 130 can be located on the leading edge which engaged the ramp 622 of the nose cone 620. The small chamfer 130 can facilitate delivery of the tissue anchor 100. In FIGS. 14D-14F, the suture tail 154 and the suture loop 160 were left loose. The tissue anchor 100 can be deflected off the ramp 622. The tissue anchor 100 can continue at an angled trajectory. The tissue anchor 100 can continue at an angled trajectory through the side opening 626 of the hypo tube 602. In some methods, the tissue anchor 100 can continue at an angled trajectory not the desired ninety degree trajectory. In FIGS. 14G-14H, the suture tail 154 and the suture loop 160 can be fixed. In FIGS. 14G-14H, the suture tail 154 and the suture loop 160 can have tension applied. The tissue anchor 100 can deflect off the ramp 622. In some methods, the tissue anchor 100 rotated ninety degree due to the constrained suture tail 154 and suture loop 160.

FIG. 141 illustrates the push rod 604. The tip of the push rod 604 can be chamfered. The chamfer 628 of the push rod 604 can assist in manipulating the tissue anchor 100. The chamfer 628 of the push rod 604 can facilitate pushing the tissue anchor completely out of the hypo tube 602. In some embodiments, the ramp 622 and the chamfer 628 are the same angle. In some embodiments, the ramp 622 and the chamfer 628 are different angles. In some embodiments, the ramp 622 and the chamfer 628 are mirror images. In some embodiments, the ramp 622 and the chamfer 628 taper inward. In some embodiments, the ramp 622 and the chamfer 628 taper away from the opening 626. The opening 626 can be spaced from the distal end of the hypo tube 602. The opening 626 can be a lateral opening of the hypo tube 602. The tissue anchor 100 can be deployed through the opening 626.

The delivery system 600 can include a ramped feature to assist in turning the tissue anchor 100. The nose cone 620 can include the ramp 622. The ramp 622 can assist in turning the tissue anchor 100. The push rod 604 can include the chamfer 628. The chamfer 628 can assist in turning the tissue anchor 100. The ramped feature can turn the tissue anchor 100 from a generally longitudinal orientation within the hypo tube 602 to a generally perpendicular orientation when deployed. The ramped feature can pivot the tissue anchor 100 from a generally longitudinal orientation within the hypo tube 602 to a generally angled orientation through the opening 626. The method can include additional deployment steps to ensure the anchor is deployed to ninety degrees. In some embodiments, tension can be applied to the suture tail 154 to ensure that the tissue anchor 100 pivots to ninety degrees. In some embodiments, tension can be applied to the suture loop 160 to ensure that the tissue anchor 100 pivots to ninety degrees. In some embodiments, an additional tensioning feature on suture can be utilized to maintain the rotational trajectory out of the tissue anchor 100. In some embodiments, two anchors are delivered. In some embodiments, the tissue anchor 100 comprises a double anchor system. In some embodiments, the double implant system creates compression with two separate tissue anchor deployments.

FIG. 14J illustrates the tissue anchor 100 in ballistic gelatine. The tissue anchor 100 can be deployed in ballistics gelatin using the same technique described herein. In some methods, with the addition of surrounding forces and pressure from the gelatin, the tissue anchor 100 encountered forces preventing the tissue anchor 100 from fully deploying from the hypo tube 602. In some methods, the tissue anchor 100 was trapped or pinched by the push rod 604 and the ramp feature 622. In some methods, the hypo tube 602 could not withdraw without pulling the tissue anchor back with the hypo tube 602.

FIGS. 15A-15F illustrate a method of deployment. The deployment method can include inserting the hypo tube 602 as shown in FIG. 15A. The hypo tube 602 can be loaded with the tissue anchor 100.

The deployment method can include advancing the push rod 604 to deploy the tissue anchor 100 as shown in FIG. 15B. The push rod 604 can push the tissue anchor 100 from the end of the hypo tube 602. The tissue anchor 100 can pivot as shown in FIG. 15C as the tissue anchor 100 is deployed. The tissue anchor 100 can be fully deployed as shown in FIG. 15D. The hypo tube 602 can be retracted as shown in FIG. 15E. The suture loop 160 can be tensioned as shown in FIG. 15F. The tissue anchor 100 resists retraction. The tissue anchor 100 can be pulled from a generally midpoint location. The tissue anchor is orientated perpendicularly. The suture tail 154 can be tensioned as shown in FIG. 15G. The tissue anchor 100 can pivot to be aligned with the delivery tract. The tissue anchor 100 can be pulled from a generally lateral location. The tissue anchor 100 can be orientated longitudinally. The tissue anchor 100 can be fully retracted by pulling the suture tail 154.

FIGS. 16A-16C illustrate features of the tissue anchor 500. The tissue anchor 500 can include any feature described herein. FIG. 16A illustrates the tissue anchor 500. The tissue anchor 500 can advantageously produce promising results. The tissue anchor 500 can reliably pivot to ninety degrees.

FIG. 16B illustrates the delivery system 600. The deployment method can include inserting the loaded hypo tube 602 into tissue. The hypo tube 602 can be loaded with the tissue anchor 500. The hypo tube can be inserted at a depth into the tissue. The hypo tube 602 can be inserted while the first suture tail 554 is held or fixed relative to the hypo tube 602. The method can include advancing the push rod 604 until the push rod 604 was flush with the tip of the hypo tube 602. In some embodiments, a mark is provided on the hypo tube 602 and/or the push rod 602 to ensure proper depth of travel each time. The method can include leaving both the first suture tail 554 and the second suture tail 561 slack while the hypo tube 602 and the push rod 604 were fully retracted. The method can include pulling the first suture tail 554 to retract the tissue anchor 500. The method can include pulling the second suture tail 561 to apply a load.

The bench test was performed using a bovine tissue and a hand held force gauge to measure peak pullout forces. The tissue anchor 500 was repeatedly delivered. Out of the trials, the maximum pullout force achieved was 5.62 kgf. There were eight trials that followed the deployment technique to yield a fully deployed anchor. FIG. 16C illustrates the bench test setup. The tissue anchor 500 can be deployed in the bovine tissue and the suture tail 554 can extend from the bovine tissue.

In two trials, the tissue anchor was deployed without a fixed suture. The tissue anchor 100 was parallel to the tract. The maximum pullout force was 1.5 kgf and 1.52 kgf when the anchor was intentionally deployed parallel to the tract. These trials set the threshold pullout force when the anchor was not fully deployed. These trials set the threshold pullout force to understand whether the tissue anchor had turned ninety degrees. Trials where the pull out force was less than 1.5 kgf suggest that the tissue anchor was not fully turned ninety degrees, such that the tissue anchor did not bite and stay oriented. These trials intentionally deployed the tissue anchor parallel to the tract without the suture line fixed to determine the threshold force.

FIGS. 17A-17L illustrate features of a tissue anchor 700. The tissue anchor 700 can be used with any assembly described herein. The tissue anchor 700 can include any of the features or methods of use of the tissue anchor assembly 10, 50 described herein. The tissue anchor 700 can be used with the suture 150, 550 described herein. The tissue anchor 700 can include an elongated member 702. The elongated member 702 can include a longitudinal axis 704. The longitudinal axis 704 can extend from a first end 706 to a second end 708. The elongated member 702 can include any elongated shape. The elongated member 702 can include a lumen 710. The lumen 710 can extend along the longitudinal axis 704. The lumen 710 can extend the entire length of the elongated member 702. The lumen 710 can extend a portion of the length of the elongated member 702, such as from the first end 706 to a midpoint along the length of the elongated member 702. The lumen 710 can extend a portion of the length of the elongated member 702, such as from the second end 708 to a midpoint along the length of the elongated member 702.

The tissue anchor 700 can include one or more tapered edges. The tissue anchor 700 can include one or more flat edges. The tissue anchor 700 can include one or more rounded edges. The first end 706 can include a tapered edge 716. The tapered edge 716 can slant downward and inward. The second end 708 can include a double tapered edge 718. The double tapered edge 718 can form a ridge. The first end 706 can be blunt. The first end 706 can be sharpened. The second end 708 can be blunt. The second end 708 can be sharpened.

The tissue anchor 700 can include an opening 720. The opening 720 can extend from the outer surface of the elongated member 702 to the lumen 710. The opening 720 can be located along the length of the elongated member 502. The opening 720 can be located at a midpoint of the elongated member 702. The opening 720 can extend from the midpoint of the elongated member 702 to the first end 706. The opening 720 can extend to the first end 706. The opening 720 can include a through axis 722. The through axis 722 can be transverse to the longitudinal axis 704. The opening 720 can be perpendicular to the lumen 710.

The tissue anchor 700 can include textured features. The tissue anchor 700 can include design features to enhance anchorage to the tissue. The tissue anchor 700 can include ribs, fins, barbs, spirals, or other features. The tissue anchor 700 can be 3D printed. The tissue anchor 700 can include longitudinal ribs 730 as shown in FIGS. 17A-17C. The longitudinal ribs 730 can be oriented along the longitudinal axis 704. The longitudinal ribs 730 can be arranged around the circumference of the tissue anchor 700. The longitudinal ribs 730 can be equally spaced. The longitudinal ribs 730 can be unequally spaced. Two or more longitudinal ribs 730 can be along a straight line. Two or more longitudinal ribs 730 can be longitudinally offset. The tissue anchor 700 can include any number of longitudinal ribs 730, for example, 2 longitudinal ribs, 3 longitudinal ribs, 4 longitudinal ribs, 5 longitudinal ribs, 6 longitudinal ribs, 7 longitudinal ribs, 8 longitudinal ribs, 9 longitudinal ribs, 10 longitudinal ribs, 15 longitudinal ribs, 20 longitudinal ribs, 25 longitudinal ribs, 30 longitudinal ribs, or any range of two of the foregoing values. The tissue anchor 700 can include undercuts 732. The undercuts 732 can form the edges of the longitudinal ribs 730. The undercuts 732 can taper inward.

The tissue anchor 700 can include rows of barbs as shown in FIGS. 17D-17F. The tissue anchor 700 can include the first row of barbs 740. The tissue anchor 700 can include a plurality of the first row of barbs 740. The first row of barbs 740 can be arranged circumferentially. The first row of barbs 740 can form a ring. The first row of barbs 740 can be arranged at a first longitudinal location. The plurality of the first row of barbs 740 can be equally spaced. The plurality of the first row of barbs 740 can be unequally spaced. The first row of barbs 740 can point toward the first end 706. The first row of barbs 740 can be slanted. The first row of barbs 740 can extend radially outward. The first row of barbs 740 can be angled relative to the elongated member 702. The plurality of the first row of barbs 740 can form multiple spaced apart rings.

The tissue anchor 700 can include the second row of barbs 742. The tissue anchor 700 can include a plurality of the second row of barbs 742. The second row of barbs 708 can be arranged circumferentially. The second row of barbs 708 can form a ring. The second row of barbs 742 can be arranged at a second longitudinal location. The plurality of the second row of barbs 742 can be equally spaced. The plurality of the second row of barbs 742 can be unequally spaced. The second row of barbs 742 can point toward the second end 708. The second row of barbs 742 can be slanted. The second row of barbs 742 can extend radially outward. The second row of barbs 742 can be angled relative to the elongated member 702. The plurality of the second row of barbs 742 can form multiple spaced apart rings.

The first row of barbs 740 and the second row of barbs 742 can point in opposite directions. The barbs of the first row of barbs 740 and the barbs of the second row of barbs 742 can be pointed. The barbs of the first row of barbs 740 and the barbs of the second row of barbs 742 can be blunt. The barbs of the first row of barbs 740 and the barbs of the second row of barbs 742 can be tapered. The barbs of the first row of barbs 740 and the barbs of the second row of barbs 742 can have the same shape. The barbs of the first row of barbs 740 and the barbs of the second row of barbs 742 can have different shapes.

The opening 720 can be located at a midpoint. The rows of barbs 706, 708 can be mirrored about the midpoint. The rows of barbs 706, 708 can be mirrored about the mid-plane. The rows of barbs 706, 708 can be mirrored about the opening 720.

The tissue anchor 700 can include rows of fins 750 as shown in FIGS. 17G-17I. The fin 750 can be continuous. In some embodiments, the fin 750 does not include perforations or breaks. The fin 750 can be arranged circumferentially. The fin 750 can form a ring. The fin 750 can be arranged at a first longitudinal location. The fin 750 can be blunt. The fin 750 can extend radially outward. The plurality of fins 750 can be equally spaced. The plurality of fins 750 can be unequally spaced. The plurality of fins 750 can form multiple spaced apart rings.

The tissue anchor 700 can include a spiral 760, 762 as shown in FIGS. 17J-17L The tissue anchor 700 can include the first spiral 760. The first spiral 760 can be arranged circumferentially. The first spiral 760 can extend toward the first end 706. The first spiral 760 can extend radially outward. The tissue anchor 700 can include a second spiral 762. The second spiral 762 can be arranged circumferentially. The second spiral 762 can extend toward the second end 708. The second spiral 762 can extend radially outward.

The first spiral 760 and the second spiral 762 can point in opposite directions. The first spiral 760 and the second spiral 762 can be sharpened. The first spiral 760 and the second spiral 762 can be blunt. The first spiral 760 and the second spiral 762 can be tapered. The first spiral 760 and the second spiral 762 can have the same shape. The first spiral 760 and the second spiral 762 can have different shapes.

The opening 720 can be located at a midpoint. The first spiral 760 and the second spiral 762 can be mirrored about the midpoint. The first spiral 760 and the second spiral 762 can be mirrored about the midplane. The first spiral 760 and the second spiral 762 can be mirrored about the opening 720.

FIGS. 17M-17N illustrate the delivery system 600. The delivery system 600 can include any feature described herein. The hypo tube 602 can include a 0.120″ outer diameter tube with an inner diameter of 0.110″. The tissue anchor 700 can be used with a suture 750. The suture 750 an include any features of the sutures described herein. FIG. 170 illustrate the bench top setup. The bovine tissue can be used for pullout testing. The deployments were made through a 0.5″ outer diameter hole in a container used to simulate bone structure.

FIGS. 17P-17Y illustrate the smallest cross section area. FIGS. 17P-17Q illustrate the tissue anchor 700 with the first spiral 760 and the second spiral 762. The area of the smallest cross-section is 3.114 mm². FIGS. 17R-17S illustrate the tissue anchor 700 with the rows of the fins 750. The area of the smallest cross-section is 2.850 mm². FIGS. 17T-17U illustrate the tissue anchor 700 with the first row of barbs 740 and the second row of barbs 742. The area of the smallest cross-section is 2.495 mm². FIGS. 17V-17W illustrate the tissue anchor 700 with the longitudinal ribs 730. The area of the smallest cross-section is 2.405 mm². The smallest cross section can occur near the opening 720. FIGS. 17X-17Y illustrates the tissue anchor 700 with a non-textured surface 770. The tissue anchor 700 with the non-textured surface 770 can have a slightly larger cross-sectional area than the textured designs. The area of the smallest cross-section is 3.537 mm². The tissue anchor 700 can have a weak point near the opening 720. The tissue anchor 700 can have a weak point at the smallest cross-section. The table below provides examples of cross-sections area compared to breaking strength for various tissue anchors. The tissue anchor with the largest cross-sectional area has the highest pullout force breakage. The tissue anchor with the smallest cross-sectional area has the smallest pullout force breakage.

| | Smallest cross | Pullout Force |
|---|---|---|
| Anchor | section area | Breakage |
| Non-textured tissue anchor 770 | 3.537 mm² | 2.82 kgf |
| Longitudinal ribs 730 | 2.405 mm² | 1.37 kgf |
| Rows of barbs 740, 742 | 2.495 mm² | 2.09 kgf |
| Fins 750 | 2.850 mm² | 1.93 kgf |
| Spiral 760, 762 | 3.114 mm² | 1.84 kgf |

FIGS. 17Z-17AA illustrate a break line example. The tissue anchor 700 with the fins 750 is illustrated. The break line can originate at the smallest cross section area. The break line can originate at the opening 720.

The tissue anchor 700 can be 3D printed. The tissue anchor 700 can comprise an engineered plastic. The tissue anchor 700 can break at the same location. The tissue anchor 700 can break at the smallest cross-section around the opening 720 along the midline where the tensioning suture exits. In some testing, the break line was diagonal. While not to be bound by a theory, the break line was diagonal because the suture running through half of the tissue anchor was the strongest and the opposing end deflected forming a ‘Y’ until breaking occurred. Since all anchors broke during pullout, this demonstrates that all deployments were properly anchored at approximately ninety degrees. The textured features provided some anchorage which prevented the tissue anchor from slipping and turning parallel to the tract. In some methods, while the breaking force may not correlate to the textured feature design, the design did dictate the smallest cross-sectional area geometry. In some embodiments, the smallest cross-sectional area can be increased to avoid breakage. In some embodiments, the smallest cross-sectional area is moved away from the midline.

FIGS. 18A-18P illustrate additional designs. The smallest cross-sectional area can be improved. FIGS. 18A-18B illustrate the tissue anchor 700 with the first spiral 760 and the second spiral 762. The area of the smallest cross-section is 3.114 mm². FIGS. 18C-18D illustrate the tissue anchor 700 with the rows of the fins 750. The area of the smallest cross-section is 3.470 mm². FIGS. 18E-18F illustrate the tissue anchor 700 with the first row of barbs 740 and the second row of barbs 742. The area of the smallest cross-section is 2.495 mm². FIGS. 18G-18H illustrate the tissue anchor 700 with the longitudinal ribs 730. The area of the smallest cross-section is 2.405 mm². FIGS. 181-18J illustrated the tissue anchor 700 with the non-textured surface 770. The tissue anchor 700 with the non-textured surface 770 has the largest cross-sectional area compared to the textured designs. The area of the smallest cross-section is 3.537 mm².

FIGS. 18K-18L illustrate the tissue anchor 700 with the fins 750. The area of the smallest cross-section is 4.147 mm². The central area near the opening 720 can be increased in diameter. The design can increase the smallest cross-sectional area to avoid breakage. The fins can be 0.010″. FIGS. 18M-18N illustrate the tissue anchor 700 with the fins 750. The area of the smallest cross-section is 4.147 mm². The central area near the opening 720 can be increased in diameter. The design can increase the smallest cross-sectional area to avoid breakage. The fins can be 0.005″. The central area can have a length of 0.100″. FIGS. 180-18P illustrate the tissue anchor 700 with the first row of barbs 740 and the second row of barbs 742. The area of the smallest cross-section is 4.147 mm². The central area near the opening 720 can be increased in diameter. The design can increase the smallest cross-sectional area to avoid breakage.

FIGS. 19A-19C illustrate a method of deployment. The tissue anchor 700 can comprise PEEK. The tissue anchor 700 can include additional design features described herein. The additional features can impact anchoring strength. The additional design features can increase the maximum pullout force. The machined PEEK anchors can have increased maximum pullout force compared to 3D printed materials. In some embodiments, the goal can be 4.0 kgf. The tissue anchor 700 can be delivered with the delivery system 600. The hypo tube 602 can include a 0.120″ outer diameter tube with an inner diameter of 0.110″.

The tissue anchor 700 with the non-textured surface 770 is illustrated in FIG. 19A. The tissue anchor 700 with the non-textured surface 770 can comprise PEEK rod. The outer diameter can be 0.098 inches. The minor diameter can be 0.098 inches. The tissue anchor 700 with the non-textured surface 770 can be cylindrical. The tissue anchor 700 with the non-textured surface 770 can have a length of 0.490 inches. The tissue anchor 700 with the non-textured surface 770 can have a diameter of the opening 720 of 0.033 inches. The outer diameter of the hypo tube 602 can be 0.120 inches. The first end 706 can be flat. The second end 708 can be flat.

The tissue anchor 700 can include fins 750 as shown in FIG. 19B. The tissue anchor 700 with the fins 750 can be machined from PEEK rod. The outer diameter can be 0.098 inches. The minor diameter can be 0.082 inches. The tissue anchor 700 with the fins 750 can have a major and minor diameter based on the height of the fins 750. The tissue anchor 700 with the fins 750 can have a length of 0.529 inches. The tissue anchor 700 with the fins 750 can have a diameter of the opening 720 of 0.033 inches. The first end 706 can be flat. The second end 708 can be flat.

The tissue anchor 700 can be used in combination with the suture 750 as described herein. The suture 750 was knotted with one overhand knot on each end and another at the midline of the tissue anchor 700. The suture 750 can be size 2 co-braided polyester fiber suture. The tissue anchors 700 were tested with bovine tissue.

FIG. 19C illustrates the bench test setup. The bovine tissue was used for pullout testing. The deployments were made through a 0.75″ outer diameter hole in a container used to simulate bone structure. The depth of insertion was approximately 2 inches. The anchors exceeded the maximum pullout force achieved by any of the 3D printed anchors. The average pullout force of 3.93-3.95 kg exceeded the maximum pullout force achieved by 3D printed anchors at 2.82 kg. The standard deviation of the pullout force was tighter than previous machined versions of Delrin.

The method included deployments with tissue grain. FIG. 19D illustrates the results with the tissue grain. Deployments were made by alternating each anchor version in the same relative location to account for bovine tissue variability and not bias one anchor version. In all trials with the tissue grain, the tissue anchor 700 pulled out and there was no breakage of the tissue anchor or the suture lines. The graph illustrates the pullout forces for the tissue anchor 700 with the non-textured surface 770 and the tissue anchor 700 with the fins 750. The average pullout force for the tissue anchor with the non-textured surface 770 was 3.95 kg with a standard deviation of 0.59. The average pullout force for the tissue anchor 700 with the fins 750 was 3.93 kg with a standard deviation of 1.21. The tissue anchor with the non-textured surface 770 and the tissue anchor 700 with the fins 750 comprised PEEK.

The method included deployments across the tissue grain. FIG. 19E illustrates results across the tissue grain. Deployments were made by alternating each anchor version in the same relative location to account for bovine tissue variability and not bias one anchor version. In all trials cross-grain, the tissue anchor 700 reached approximately 6 kg and the tension was stopped to avoid breakage. There was no breakage of the tissue anchor 700. The graph illustrates the pullout forces for the tissue anchor 700 with the non-textured surface 770 and the tissue anchor 700 with the fins 750. The average pullout force for the tissue anchor 700 with the non-textured surface 770 was 6.24 kg with a standard deviation of 0.13. The average pullout force for the tissue anchor 700 with the fins 750 was 6.1 kg with a standard deviation of 0.08. The tissue anchor 700 with the non-textured surface 770 and the tissue anchor 700 with the fins 750 comprised PEEK.

FIG. 19F illustrates a trial with the tissue anchor 700 with the fins 750. The tissue anchor 700 is deployed cross-grain. The suture 750 broke at the overhand knot. There were approximately 9 deployment cycles on this suture line. The bovine tissue was carefully dissected down to the tissue anchor 700 to observe the deployment position. The tissue anchor 700 was deployed perpendicular to the deployment tract. The suture line vector in yellow is also illustrated.

In comparison with tissue deployments with grain versus cross-grain, the deployments with the cross-grain peak loads exceeded 6 kg and the deployments with grain pulled out on average about 4 kg. The cross-grain tissue experienced an increase of at least 2 kg of anchorage.

FIGS. 20A-20E illustrate the hypo tube 602 of the delivery system 600 and a nose cone 630. The hypo tube 602 can be a delivery tube. The hypo tube 602 can include a tapered tip. The hypo tube 602 can include an oblong opening. The hypo tube 602 can incorporate the nose cone 630. The nose cone 630 can be crimped into the end of the hypo tube 602. The nose cone 630 can be coupled to the hypo tube 602 through adhesive, welding, one or more fasteners, one or more rivets, clinching, brazing, or other joining means. The nose cone 630 and the hypo tube 602 can be integrally or monolithically formed. The nose cone 630 can comprises plastic. The nose cone 630 can be coupled to the tip of the hypo tube 602. The nose cone 630 can prevent accidental pass through the posterior wall of the tongue. FIG. 20A illustrates an isometric view of the nose cone 630. FIGS. 20B-20D illustrate side views of the nose cone 630. FIG. 20E illustrates an end view of the nose cone 630.

FIGS. 21A-21F illustrate features of a delivery system 800. The delivery system 800 can include any of the features of the delivery systems described herein. The delivery system 800 can deliver any tissue anchor described herein. The delivery system 800 can improve tensioned line retention. As described herein, the delivery system 600 can rely on a user to pull the suture line into the suture cleat 608. In some methods, the delivery system 600 worked with high forces, but was variable based on how hard the suture 150 was pulled into the suture cleat 608. In some methods, for the delivery system 600, the harder the suture 150 was secured in the suture cleat 608, the more difficult the suture 150 was to release. In some methods, the user may forget to release the suture 150 from the suture cleat 608, causing the tissue anchor to dislodge. In some methods, the suture cleat 608 can be static. The handle of the delivery system 600 can include the suture cleat 608.

The delivery system 800 can include a suture trap 840. The suture 150, 550, 750 can be tensioned. The tensioned suture 150, 550, 750 is pulled and laid in a track 842 while the suture trap 840 is pushed down. The suture trap 840 can wedge the suture 150, 550, 750 into a tortuous path. The suture trap 840 can be pivoting. The suture trap 840 can pivot from an open configuration to a closed configuration. In the open configuration, the suture 150, 550, 750 can be placed in the track 842. The track 842 can be linear. The track 842 can be nonlinear. The track 842 can be angled. The track 842 can be curved. The track 842 can be tortuous. The suture trap 840 can be pivoted closed. In the closed configuration, the suture 150, 550, 750 can be retained in the track 842. FIG. 21A illustrates the delivery system 800 with the suture trap 840 in the closed configuration as viewed from the side. FIG. 21B illustrates the delivery system 800 with the suture trap 840 in the closed configuration as viewed from the front. The suture trap 840 is pushed down. FIG. 21C illustrates the delivery system 800 with the suture trap 840 in the open configuration.

The delivery system 800 can include a hypo tube 802. The hypo tube 802 can have a small diameter such as a 0.120 inch outer diameter. The delivery system 800 can include a push rod 804. The push rod 804 can be at least partially disposed within the hypo tube 802. The hypo tube 802 can be sized to receive the tissue anchor 100, 500, 700. The longitudinal axis 104, 504, 704 of the tissue anchor 100, 500, 700 can align with the longitudinal axis of the delivery system 800.

The delivery system 800 can include a hypo tube handle 806. The hypo tube handle 806 is coupled to the hypo tube 802. The hypo tube handle 806 can include larger finger grips. The hypo tube handle 806 can include an ergonomic design. The hypo tube handle 806 can include the suture trap 840. The hypo tube handle 806 can include the track 842. The suture trap 840 can receive the suture 150, 550, 750. The suture trap 840 can receive the suture tail 154, 554. The suture trap 840 can receive the suture loop 160. The suture trap 840 can receive the second suture tail 561. In the illustrated embodiment, the suture trap 840 receives the suture tail 154 of the suture 150. The delivery system 800 can include a push rod handle 812. The push rod handle 812 is coupled to the push rod 804 to control longitudinal movement of the push rod 804. The delivery system 800 can omit the stop 610.

The push rod handle 812 can bottom out to fully deploy the tissue anchor 100, 500, 700. The push rod handle 812 can bottom out against the hypo tube handle 806. The push rod handle 812 can bottom out at the end of travel of the push rod 804. The push rod handle 812 can bottom out at a predetermined distance. The push rod handle 812 can abut the hypo tube handle 806 when the tissue anchor 100, 500, 700 is deployed from the hypo tube 802.

The push rod handle 812 can abut the suture trap 840 to fully deploy the tissue anchor 100, 500. The push rod handle 812 can bottom out against the suture trap 840. The push rod handle 812 can bottom out to pivot the suture trap 840. The push rod handle 812 can bottom out to release the suture 150, 550, 750. The push rod handle 812 can abut the suture trap 840 to passively release the suture 150, 550, 750. The push rod handle 812 can abut the suture trap 840 to avoid user error in forgetting to release the suture 150, 550, 750 from the suture trap 840. The push rod handle 812 can abut the suture trap 840 to avoid the tissue anchor 100, 500, 700 dislodging due to user error. The push rod handle 812 can abut the suture trap 840 when the tissue anchor 100, 500, 700 is deployed from the hypo tube 802. The push rod handle 812 can abut the suture trap 840 to passively release tension on the suture 150, 550, 750. The push rod handle 812 can abut the suture trap 840 to passively ensure that tissue anchor 100, 500, 700 remains lodged in tissue.

The delivery system 800 was tested in ballistic gelatin. The testing demonstrated that the tensioned suture line 150, 550, 750 within the suture trap 840 remained in tension during the deployment. The testing demonstrated that the tensioned suture line 150, 550, 750 within the suture trap 840 remained in tension forcing the tissue anchor 100, 500, 700 to turn. When the push rod handle 812 bottoms out relative to the hypo tube handle 806, the push rod handle 812 bumps the pivoting suture trap 840 out of the way and releases the tensioned suture line 150, 550 passively. FIG. 21C illustrates the push rod handle 812 bottomed out, thereby pivoting the suture trap 840 open. The push rod handle 812 bumps the suture trap 840, thereby pivoting the suture trap 840 to release the suture tail 154.

FIGS. 21D-21F illustrate an additional concept to retain the suture 150, 550, 750. The delivery system 800 can include a spring loaded trap 850. The spring loaded trap 850 can include a cantilever. The delivery system 800 can include a sailing rope cleat 860. The suture trap 840 may secure the suture better than the spring loaded trap 850. The suture trap 840 may secure the suture better than the sailing rope cleat 860. The suture trap 840 may be less complex than the spring loaded trap 850. The suture trap 840 may be less complex than the sailing rope cleat 860. The suture trap 840 can include the passive release. The suture trap 840 may have advantages over other designs.

The delivery system 800 can include improved suture retention. The delivery system 800 can eliminate the step of releasing the fixed tensioned suture prior to delivery system extraction. The delivery system 800 can passively release the suture 150, 550, 750. The delivery system 800 can passively release the suture 150, 550, 750 to avoid user error in forgetting to release the suture 150, 550, 750. The delivery system 800 can ensure perfect deployment of the tissue anchor 100, 500, 700.

FIGS. 21G-21H illustrate the delivery system 800. The delivery system can include a latch feature. The delivery system can include a stop buffer. The stop buffer can be the space between the push rod handle 812 and the hypo tube handle 806. The suture trap 840 can include a latch feature 844. The push rod handle 812 can include a corresponding latch feature 846. The latch feature 844 and the corresponding latch feature 846 secure the suture trap 840 and the push rod handle 812. The latch feature 844 and the corresponding latch feature 846 provide a secure point of contact to hold the suture trap 840 and the push rod handle 812 together.

The latch feature 844 of the suture trap 840 and the corresponding latch feature 846 of the push rod handle 812 act as a stop. The latch feature 844 of the suture trap 840 and the corresponding latch feature 846 of the push rod handle 812 can prevent movement of the push rod 804 when inserting the delivery system 800 into tissue. The latch feature 844 of the suture trap 840 and the corresponding latch feature 846 of the push rod handle 812 can prevent accidental deployment of the tissue anchor 100, 500, 700 when inserting the delivery system 800 into tissue. The latch feature 844 and the corresponding latch feature 846 allow the delivery system to move as a single unit during deployment. The latch feature 844 and the corresponding latch feature 846 prevent movement of the push rod 804 relative to the hypo tube 802 during deployment.

The delivery system 800 can be delivered to tissue. The hypo tube 802 can be delivered to a target location. The push rod handle 812 can be pushed forward. The corresponding latch feature 846 of the push rod handle 812 can slide past the latch feature 844 of the suture trap 840. The corresponding latch feature 846 of the push rod handle 812 can slide past the latch feature 844 under force by the user. The suture trap 840 can include the guide edge 848. The corresponding latch feature 846 of the push rod handle 812 can slide along the guide edge 848. The corresponding latch feature 846 of the push rod handle 812 can slide along the guide edge 848 to pivot the suture trap 840. The corresponding latch feature 846 of the push rod handle 812 can slide along the guide edge 848 to release the suture 150, 550, 750.

FIGS. 211-21L illustrate a method of loading the delivery system 800. FIG. 211 illustrates the tissue anchor 100, 500, 700 is inserted into end of hypo tube 802. The tissue anchor 100, 500, 700 is coupled to the suture 150, 450, 750. The tissue anchor 100 and the suture 150 are illustrated. The suture 150 can include the suture tail 154 and the suture loop 160. The suture tail 154 can be a long single tension line. The suture tail 154 can extend from the distal end of the tissue anchor 100. The suture tail 154 can extend from the distal end of the hypo tube 602 when the tissue anchor 100 is loaded.

FIG. 21J illustrates the suture tail 154 is placed in the track 842. The track 842 extends as a groove along the hypo tube handle 806. The track 842 can be diagonal. The track 842 can extend from a proximal end of the hypo tube handle 806 to a distal end of the hypo tube handle 806. The track 842 can receive the suture tail 154. The user can apply tension to the suture tail 154.

FIG. 21K illustrates the suture trap 840. The suture trap 840 can be pressed down by the user. The suture trap 840 can be pressed down toward the hypo tube handle 806. The suture trap 840 can be closed. The suture trap 840 can tension the suture tail 154. The suture trap 840 can secure the suture tail 154 in the track 842.

FIG. 21L illustrates the delivery system 800 prepared for deployment. The push rod handle 812 can be advanced toward the hypo tube handle 806. The latch feature 844 of the suture trap 840 and the corresponding latch feature 846 of the push rod handle 812 can be engaged. The corresponding latch feature 846 of the push rod handle 812 can maintain tension on the suture trap 840. The corresponding latch feature 846 of the push rod handle 812 can maintain tension on the suture 150. The corresponding latch feature 846 of the push rod handle 812 can maintain tension on the suture tail 154 in the track 842. The hypo tube 802 can be delivered to the target location. The push rod 804 can be prevented from movement as the hypo tube 802 is delivered via the latch feature 844 of the suture trap 840 and the corresponding latch feature 846 of the push rod handle 812. To deploy the tissue anchor 100, the push rod 804 can be advanced. The push rod handle 812 is advanced, thereby releasing the latch feature 844 of the suture trap 840. The push rod handle 812 can be advanced and the corresponding latch feature 846 of the push rod handle 812 can slide along the guide edge 848. The tension on the suture tail 154 can be passively released as the push rod handle 812 is advanced. The tissue anchor 100 can be fully deployed. The tissue anchor 100 can pivot to be generally perpendicular to the hypo tube 802. The tissue anchor 100 can pivot to be generally perpendicular to the delivery tract.

The delivery system 800 is easy to use. The delivery system 800 can allow for tissue anchor delivery that is easy to perform and yields predictable results. The delivery system 800 can avoid user error by passively releasing the suture tail 154. The delivery system 800 can be a repeatable and easy to use system. The tissue anchor 100, 500 can be easy to load and deploy. The delivery system 800 can be 3D printed.

FIG. 22 illustrates multiple tissue anchors. The tissue anchors can be delivered with any delivery system described herein. The hypo tube 602, 802 can have a diameter of 0.145″. The tissue anchor 100 can have an outer diameter of 0.110″. The tissue anchor 100 can have a length of 0.50″. The tissue anchor 100 can comprise PEEK. The suture 150 can be a size 2 suture. The suture 150 can be co-braided suture. The tissue can be bovine tissue. FIG. 22 illustrates four variations of chamfer angle of the ends of the tissue anchor 100. The tissue anchors 100 were tested for pull out force compared with the control. The control can be stock suture. In some methods, the suture 150 must be held tight throughout the entire deployment. In some methods, the suture 150 must be held tight to ensure that the tissue anchor 100 rotates to ninety degree. In some methods, the suture 150 must be held tight by holding the suture tail 154. In some methods, the suture 150 must be held tight by holding the suture loop 160. The average pullout force for the chamfer angle design A was 2.64 kgf. The average pullout force for the chamfer angle design B was 2.83 kgf. The average pullout force for the chamfer angle design C was 3.14 kgf. The average pullout force for the chamfer angle design D was 2.53 kgf. The average pullout force for control was 3.51.

Various other modifications, adaptations, and alternative designs are of course possible in light of the above teachings. Therefore, it should be understood at this time that the inventions may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the inventions are susceptible to various modifications, and alternative forms, specific examples thereof have been shown in the drawings and are herein described in detail. It should be understood, however, that the inventions are not to be limited to the particular forms or methods disclosed, but to the contrary, the inventions are to cover all modifications, equivalents, and alternatives falling within the spirit and scope of the various embodiments described. Any methods disclosed herein need not be performed in the order recited. The methods disclosed herein include certain actions taken by a practitioner; however, they can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

1. A tissue anchor assembly comprising:
a tissue anchor comprising an elongated member, wherein the elongated member comprises a first end and a second end, wherein the elongated member comprises a lumen extending from the first end to the second end, wherein the elongated member comprises an opening between the first end and the second end; and
a suture, wherein the suture enters the lumen at the first end of the elongated member, forms a suture loop through the opening of the elongated member, and exits the lumen at the second end of the elongated member.

2. The tissue anchor assembly of claim 1, wherein the suture is a continuous suture.

3. The tissue anchor assembly of claim 1, wherein the suture comprises a suture tail extending from the first end of the elongated member.

4. The tissue anchor assembly of claim 3, wherein the suture tail is configured to act as the release suture to remove the tissue anchor.

5. The tissue anchor assembly of claim 1, further comprising a bone anchor.

6. The tissue anchor assembly of claim 5, wherein the suture loop is configured to be secured to the bone anchor.

7. The tissue anchor assembly of claim 5, wherein the bone anchor is configured to be implanted on a mandible of a patient.

8. The tissue anchor assembly of claim 1, wherein the suture comprises a knot near the second end of the elongated member.

9. The tissue anchor assembly of claim 8, wherein the knot is an overhand knot.

10. The tissue anchor assembly of claim 1, wherein the suture comprises a knot near the first end of the elongated member.

11. The tissue anchor assembly of claim 10, wherein the knot is an overhand knot.

12. The tissue anchor assembly of claim 1, wherein the suture comprises a knot along the suture loop.

13. The tissue anchor assembly of claim 12, wherein the knot is an overhand knot.

14. The tissue anchor assembly of claim 12, wherein the knot is spaced from the opening of the elongated member.

15. A tissue anchor delivery system comprising:
a tissue anchor comprising an elongated member and a suture;
a hypo tube handle;
a hypo tube coupled the hypo tube handle, wherein the hypo tube is configured to receive the elongated member;
a push rod handle;
a push rod coupled to the push rod handle, wherein the push rod is disposed at least partially within the hypo tube, wherein the push rod is configured to advance the elongated member from the hypo tube; and
a stop disposed between the hypo tube handle and the push rod handle.

16. The tissue anchor assembly of claim 15, wherein the hypo tube handle comprises a suture cleat.

17. The tissue anchor assembly of claim 15, wherein the stop is configured to be removed to allow the push rod handle to advance toward the hypo tube handle to advance the elongated member from the hypo tube.

18. A method comprising:
advancing an elongated member of a tissue anchor into tissue, wherein the elongated member is coupled to a suture, wherein the suture enters a lumen at a first end of the elongated member, forms a suture loop through an opening between the first end and a second end of the elongated member, and exits the lumen at the second end of the elongated member; and
securing the suture loop to a bone anchor.

19. The method of claim 18, further comprising securing the bone anchor to a mandible of a patient.

20. The method of claim 18, wherein a suture tail extending from the first end of the elongated member acts as a release suture to remove the tissue anchor.

21-46. (canceled)
